(19) United States
(12) Patent Application Publication
(10) Pub. No.: US 2021/0074178 A1
(43) Pub. Date: Mar. 11, 2021

(54) A SUBJECT-TAILORED CONTINUOUSLY DEVELOPING RANDOMIZATION BASED METHOD FOR IMPROVING ORGAN FUNCTION

(71) Applicant: OBERON SCIENCES ILAN LTD., Kfar Tavor (IL)

(72) Inventors: Yaron ILAN, Kefar Tavor (IL); Tahel ILAN BER, Hod Hasharon (IL)

(21) Appl. No.: 16/761,301

(22) PCT Filed: Nov. 4, 2018
(86) PCT No.: PCT/IL2018/051171

§ 371 Date: May 4, 2020

Related U.S. Application Data
(60) Provisional application No. 62/581,722, filed on Nov. 5, 2017.

(51) Int. Cl. G09B 19/00; G06N 20/00; G16H 20/30; G16H 20/60; A61B 5/00

(52) U.S. Cl. CPC G09B 19/0038; G06N 20/00; G16H 20/30; G09B 19/0092; A61B 5/7264; A61B 5/486; G16H 20/60

(57) ABSTRACT

The present disclosure provides systems regimens, devices and methods for improving organ function by challenged-exercise, training, and/or education and/or nutritional regimens, or devices intended for improving organ performance, and for prevention and treatment of loss of an effect to exercise regimens in healthy and chronic subjects, or lack of full responsiveness to exercise, training, nutritional or education regimens in subjects who wish to improve the function of their organs, or with chronic diseases. There are provided herein devices, systems, and methods for real time or delayed altering of the parameters of training regimens and/or time of administration and/or combining different exercise, training, nutrition or education regimens, for improving the long term effect of the regimen. According to some embodiments, any training regimen and/or device-generated maneuver/stimulation, wherein the parameters are updated within the exercise regimen/maneuver period, for personalizing the regimen parameters and increasing the accuracy and efficacy of the regimen for achieving the desired physiological goal, and to prevent long-term adaptation for ensuing prolong effect of the training regimen on the target organ function or physiological pathway. Output parameters are continuously, semi continuously, or conditionally being updated based on measurements and inputs provided to a compute circuitry configured to facilitate closed loop machine learning capabilities.

### TECHNICAL FIELD

The present disclosure generally relates to the field of improving organ function by improved challenge-based training.

### BACKGROUND

Improving the function of organs requires in many cases exercise, training, teaching, and/or education regimens for the organ as well as changes in nutrition. Many of these regimens are based on inducing challenges, which are specific for the organ. Induction of challenges to an organ can improve its function to a certain extent due to the adaptation effect of the target organ to the challenge. These challenges commonly follow set regimens intended to affect a physiological or a pathological change. They are carried out based on predetermined protocols, within a certain range, such that once a certain mode of exercise, training, teaching or education, or a challenging protocol is prescribed/configured, it is not changed until the exercise is finished or non-responsiveness occurs. Moreover, a plateau effect occurs for most exercise regimens with minor benefit following continuous use of a constant regular/perpetual training regimen. This is the case when trying to improve muscle, heart, lung, brain, or any other type of an organ. It is also the case when implying any type of exercise or challenge to improve any target organ function whether its function is normal or abnormal, such as in healthy subjects or athletes performing sport activities, and in patients with chronic heart failure, chronic lung diseases, Alzheimer's disease, and such.

While exercise, training, teaching, education, and induction of challenges for improvement of organ function show efficacy in some cases, it often reaches a “maximal plateau” for the subject. In many cases it is difficult to improve beyond that point. Exercising, training, and/or education may also be only minimally effective or not effective at all for some subjects. This is due to adaptation processes that occur in the body and/or in the target organ to prolonged exposure to the exercise or challenge, or development of any type of tolerance, prohibiting maximal effect and long lasting effect. There is thus a need in the art for more effective training and nutritional regimens that take into consideration the variability between subjects and their physiological reaction to various types of exercises, training, education and/or organ-specific challenges, and the loss of an effect or maximal response to any type of a procedure or maneuver, which is aimed at improving organ performance.

Common exercising tasks such as sport activities, learning and education tasks such as studying mathematics, languages, using flight and/or driving simulators, and treatment of chronic diseases including arthritis, asthma, chronic lung disease, heart failure, or any muscle or neurological disease, or diseases that affect brain function, may benefit from a challenge-based training. However, in most cases, adaptation occurs to the challenge, prohibiting an ability to further improve the organ(s) function/improve the task performance, and preventing the subject from reaching the maximal effect which can be achieved with the exercise. Therefore, the adaptation prevents the subject from reaching the maximal possible performance.

Most subjects undergoing any type of sports activity or any type of exercise for improvement of organ function, or any type of training, education or teaching session, or any type of nutritional change, require long term challenge-based regimens and long term focus on nutrition. For many of these, loss of the maximal effect or even most of the effect, or reaching a point where no further significant improvement can be achieved, may occur following a certain period, which is associated with several adaptation mechanisms.

There are many possible causes for losing the positive effects of exercise or of reaching a maximal effect from which no further gain is achieved by continued exercise or by greater challenges to the organ, some of which are not well understood. Some causes may involve subcellular, cellular or organ and/or inter-organ types of adaptation. Lack of ability to reach a maximal effect, and/or prolonged time required for reaching a maximal benefit from a training regimen is associated with any type of adaptation.

Most exercises, training or education sessions are designed to be based on regular and repetitious regimens. As such, they are associated with adaptation of the target organ to the exercise. The adaptation and habituation to the exercise can be at the molecular, cellular, or whole organ level or at a higher level involving the associations between several organs. It depends on factors associated with the subject's genetic, physiological background, diseases, concomitant medications and other genetic, phenotypic, and environmental factors, such that for every subject a different scheme may lead to different effects of similar stimuli and exercises.

Moreover, as most exercise, training or education regimens are based on regular regimens, or on constant, repetitious, perpetual intervals, and most are being developed on gradual stepwise schemes, such as by increasing difficulty in a gradual mode, which is associated with subject-specific adaptation, gradualness per se does not enable overcoming adaptation. It also does not enable tailoring the exercise for the subject, and in most cases is based on “one-regimen for all”.

Part of the adaptation of the target organ has to do with the brain-target organ connections. A regular or a perpetual-gradual exercise regimen, in most cases disconnects the brain from the target organ, such as when doing a monotonic sport activity which activates one muscle group.

For many chronic types of stimuli and exercises, adaptation of the target organ prohibits reaching maximal effect of the exercise, or is associated with partial or even total loss of the effect of the training regimen. Adaptation develops to some types of regimens much more rapidly than to others. The extent of adaptation or tolerance depends on the individuals' genetic, phenotypic, and other factors, as well as on the type and/or the duration of the training. Adaptation may occur within a relatively short period of time, and contribute to non-effectiveness, or minimal efficacy to any exercise, training, education, or any maneuver.

Below are several examples of adaptation, lack of ability to reach maximal effect of a training or nutrition regimen, or of partial loss of effect of maneuvers or exercises following prolonged administration:

a. Physical activity and metabolic equivalents: Physical activity performed at more than the minimum recommended level helps increase longevity. The increase in longevity however begins to plateau at approximately 300 minutes of brisk walking per week. (Leisure Time Physical Activity of Moderate to Vigorous Intensity and Mortality: A Large Pooled Cohort Analysis, Moore S C, Patel A V, Public Library of Science November 2012, e1001335). Exercise physiologists practically universally accept the Metabolic Equivalent (MET) system to express energy expenditure in relation to body weight. The American College of Sports Medicine has defined light, moderate, and heavy physical activity to equate with specific MET levels. Tables have been developed to enable prescription of exercise intensity. Tables of MET values for a variety of activities are based largely on measurements in adults but are not subject-tailored and only provide averages from diverse populations. Moderate-to-vigorous activities require about 5 to 8 METs, and such intensity is needed to derive most health benefits. However, the available data does not enable tailoring the exercise regimen to the subject. A growing body of evidence points to the fact that the MET system is inaccurate in its estimation of physical activity energy expenditure in people of different body mass and body fat percentage. It is also advised that, when calculating the energy cost of physical activities, the MET system does not take into account individual differences, nor does it consider changes in the same subject over time (Metabolic equivalent: one size does not fit all, N M Byrne, A P Hills, Journal of Applied Physiology September 2005, Vol. 99 no. 3, 1112-1119).

b. School age youth activity: School-age youth are encouraged to participate every day in 60 minutes or more of moderate to vigorous physical activity that is enjoyable and developmentally appropriate. Interventional studies indicate specific amounts of physical activity necessary for beneficial changes in the skeletal health, aerobic fitness, and muscular strength and endurance of youth, and in adiposity in youth who are overweight (Evidence Based Physical Activity for School-age Youth. William B. Strong, Journal of Pediatrics June 2005, Volume 146, Issue 6, Pages 732-737). Most programs use regimens of continuous, moderate to vigorous activities for 30 to 45 minute's duration, 3 to 5 days per week. However, none of these programs were able to find a way to tailor the regimen to the subject. It was shown that allowing for inter- and intra-individual differences in physical activity and in response to physical activity among children and adolescents, is required for achieving a better effect. Moreover, as all exercise and training programs are based on a predefined regimen, they do not accommodate changes in subject status. They also do not enable overcoming a “plateau effect” which is reached by most participants. Physical activities of children and adolescents vary with age, type of exercise, and setting. With growth, maturation, and experience, basic movements are integrated and coordinated into more specialized and complex movement skills which are different between subjects. None of the available regimens accommodate themselves to the subjects' parameters, and none of them are altered along the regimen with changes that occur during exercise. Similarly, structure and function are approached or attained in late adolescence (age 15-18 years) and in adults, in whom physical activity programs are even more structured; however, none of them are subject-tailored. Recommended priorities for physical activities during childhood and adolescence should be designed to be relative to the development of skills and to behavioral, health, and fitness benefits, subject demographic, concomitant diseases, and to many additional genetic/phenotypic/environmental factors. However, currently there is no regimen which can provide such a method for tailoring the exercise regimen to a subject. For example, during preschool and early school ages, general movement activities develop movement patterns and skills. As these basic movements become established and skills improve, health, fitness, and behavioral components of physical activities increase. Health-related activities include those that emphasize cardiovascular and muscular endurance and muscular strength and those that involve weight bearing. Similar changes occur in adults. The degree of physical activity is important in achieving positive behavioral outcomes. Therefore, there is an unmet need for refinement of the regimen to the subject (CM Malina. Fitness and performance: adult health and the culture of youth, new paradigms? In: R. J. Park and M. H. Eckert, editors. New possibilities, new paradigms? (American Academy of Physical Education Papers No. 24; Champaign, Ill.: Human Kinetics Publishers; 1991. p. 30-8). Increasing activity by 10% per week was suggested as an approach used in athletic training. However, using methods of a gradual and stepwise approach based on predetermined perpetual regimens are not subject-tailored and do not lead to continuous improvement in the target effect. Moreover, most of these regimens require prolonged exercising time and do not enable shortening the training time without jeopardizing the results of the exercise. Adherence to these programs is low.

c. Recommendations for Physical Activity: World Health Organization (WHO) recommendations for physical activity: In adults aged 18-64, physical activity includes leisure time physical activity (for example: walking, dancing, gardening, hiking, swimming), transportation (e.g. walking or cycling), occupational (i.e. work), household chores, playing, games, sports or planned exercise, in the context of daily, family, and community activities in order to improve cardiorespiratory and muscular fitness, bone health, reduce the risk of NCDs and depression (http://www.who.int/dietphysicalactivity/factsheet_adults/en/). The U.S. Department of Health and Human Services (HHS) has released physical activity guidelines for all Americans aged 6 and older (NIH recommendations https://www.nhlbi.nih.gov/health/health-topics/topics/phys/recommend). The “2008 Physical Activity Guidelines for Americans” explains that regular physical activity improves health. They encourage people to be as active as possible. These guidelines recommend the types and amounts of physical activity for children, adults and older adults, and provide tips on how to fit physical activity into daily life. Activities should vary and be a good fit for their age and physical development. No method to tailor the activity for age or subject genotype/phenotype is currently available. No method to shorten the time of activity or to improve adherence without jeopardizing the effect exists. Physical activity is recommended to be moderate-intensity aerobic activity. Examples include: walking, running, skipping, playing on the playground, playing basketball, and biking. Vigorous-intensity aerobic activity is recommended to be included at least 3 days a week. Muscle-strengthening activity is recommended to be included at least 3 days a week. Examples include playing on playground equipment, playing tug-of-war, and doing pushups and pullups. Bone-strengthening activities are recommended to be included at least 3 days a week. Examples include: hopping, skipping, jumping jacks, playing volleyball and working with resistance bands. However, none of these methods are subject-tailored. It is recommended that inactive adults should gradually increase their level of activity. However, there is no method to overcome adaptation to a gradual and perpetual stepwise approach. People gain health benefits from as little as 60 minutes of moderate-intensity aerobic activity per week. For major health benefits, at least 150 minutes of moderate-intensity aerobic activity or 75 minutes of vigorous-intensity aerobic activity per week is needed. Another option is to do a combination of both. A general rule is that 2 minutes of moderate-intensity activity counts the same as 1 minute of vigorous-intensity activity. However, it may not be the case for every subject. It is recommended that when doing aerobic activity, it should be done for at least 10 minutes at a time and be spread throughout the week. Muscle-strengthening activities that are of moderate or vigorous intensity should be included 2 or more days a week. These activities should work all of the major muscle groups (legs, hips, back, chest, abdomen, shoulders, and arms). Examples include lifting weights, working with resistance bands, and doing sit-ups and pushups, yoga, and heavy gardening. These activities, however, are not subject-tailored. Older adults above 65 years should be physically active. Older adults who do any amount of physical activity gain some health benefits. If inactive, older adults should gradually increase their activity levels and avoid vigorous activity at first. If they can't do 150 minutes of activity each week, they should be as physically active as their abilities and conditions allow. It is recommended for adults to do balance exercises if at risk for falls. Examples include: walking backwards or sideways, standing on one leg, and standing from a sitting position several times in a row.

CDC recommendations for older people are based on the following: “How do you know if you're doing moderate or vigorous aerobic activity? On a 10-point scale, where sitting is 0 and working as hard as you can is 10, moderate-intensity aerobic activity is a 5 or 6. It will make you breathe harder and your heart beat faster. You'll also notice that you'll be able to talk, but not sing the words to your favorite song. Vigorous-intensity activity is a 7 or 8 on this scale. Your heart rate will increase quite a bit and you'll be breathing hard enough so that you won't be able to say more than a few words without stopping to catch your breath. You can do moderate- or vigorous-intensity aerobic activity, or a mix of the two each week. Intensity is how hard your body is working during aerobic activity. A rule of thumb is that 1 minute of vigorous-intensity activity is about the same as 2 minutes of moderate-intensity activity. Everyone's fitness level is different. This means that walking may feel like a moderately intense activity to you, but for others, it may feel vigorous. It all depends on you-the shape you're in, what you feel comfortable doing, and your health condition. What's important is that you do physical activities that are right for you and your abilities. Besides aerobic activity, you need to do things to make your muscles stronger at least 2 days a week. To gain health benefits, muscle-strengthening activities need to be done to the point where it's hard for you to do another repetition without help. A repetition is one complete movement of an activity, like lifting a weight or doing one sit-up. Try to do 8-12 repetitions per activity that count as 1 set. Try to do at least 1 set of muscle-strengthening activities, but to gain even more benefits, do 2 or 3 sets. The activities you choose should work all the major muscle groups of your body (legs, hips, back, chest, abdomen, shoulders, and arms)”.

While there is a need for subject-tailored exercising, none of these recommendations provide a patient-tailored regimen, and none can overcome the adaptation of the organs to the repetitious perpetual training program. Currently there are no subject-tailored regimens and no methods for overcoming ongoing adaptation during exercising and training which enable reducing time of exercise, improving adherence to training regimens, and continuously improving end results.

d. Achieving maximal effect in professional sport: Professional training is based on predetermined regimens. (Training Routines for Olympic Track Sprinters:http://www.livestrong.com/article/467983-training-routines-for-olympic-track-sprinters). Good reserves of muscle glycogen are critical for Olympic sprinters, thus weight training, plyometrics and optimal nutrition are necessary to obtain world class results. Special emphasis is placed on training the quadriceps, glutes, hamstrings and calves along with a strong core to help stabilize movement. At a world class level, every athlete is different and requires an individualized workout. However, there is no method for such individualization. Sprinters have to sprint in practice, explode from the starting blocks, run in a straight line for 100 m, make any turns perfectly, accelerate into the tape at the end, and do all this with maximum efficiency. Sprinters on the USA Olympic team spend their training divided among running to build cardio capacity, strength-training to build muscle, plyometrics to increase range of motion and explosiveness, and rest time. Sprinters spend many practices running at half and three-quarter pace, in repetitive sets. A typical practice is dynamic warm-up, a lap or two to loosen up, stair runs, and then sets. Olympic hopefuls will spend the day practicing up to three separate times, with meal and rest breaks. Olympic sprinter workouts incorporate strength-training at least two days per week. Core strength and stability are just as important as leg strength. In the offseason, many sprinters lift heavier weights to build muscle. Three sets of eight to 10 repetitions are common, and during the season the emphasis is on lighter weights with higher repetitions, such as three to four sets of 15 repetitions. Most sprinters do not run on the track on weight days, or only lightly. Because Olympic sprinters need long legs, box jumping is popular as is jumping rope, skipping, and hopping through a pattern to build ankle strength. Plyometric workouts are usually performed as part of the warm-up on the track, or in the weight room. Dynamic stretching is part of every warm-up, and static stretching during cool down.

As organ adaptation varies among different athletes, it is associated with an almost “plateau effect” in their maximal achievement from which additional training may add only marginal benefits. The claim is made that every subject should have a specific algorithm to overcome his type of adaptation. There is still a need in the art for subject-tailored methods for improving organ function and overall performances in sports.

e. Complications during exercise such as asthma and stress fractures: Regular physical activity can strengthen the lungs of people who have asthma and improve their overall level of fitness. Exercise and sports can also reduce asthma symptoms. It is important to keep asthma under control and adapt physical activities to individual levels of fitness. (https://www.ncbi.nlm.nih.gov/pubmedhealth/PMH0072701/; (https://www.ncbi.nlm.nih.gov/pmc/articles/PMC4653278/). The prevalence of exercise-induced asthma is higher among elite athletes than in the general population. In elite endurance athletes, respiratory epithelial damage and reduced repair are central to the development of inflammation in the athlete's asthma phenotype. Epithelial damage is the final result of repeated courses of intensive training sessions, and of competitions at the intensity of hyperpnoea necessitated by exercise at an elite level. Airway hyper-responsiveness (AHR) is associated with periods of intensive training. In addition, when other stimuli contribute to this process by causing increased epithelial damage and inflammation, such as respiratory virus infections, AHR further develops and asthma symptoms may appear in previously asymptomatic athletes, as demonstrated by increased AHR for prolonged periods of time after respiratory virus infections. algorithm-based subject tailored-training can prevent this type of complication. There is still a need in the art for subject-tailored methods that can help prevent epithelial damage reaching the “asthma” level in professional athletes and other complications such as fractures.

f. Prevention of stress fractures during exercise: Stress fractures can occur during uncontrolled exercise or during controlled exercise when the subjects are not aware of their condition. (Lancet Volume 348, Issue 9038, 16 Nov. 1996, Pages 1343-1347 Randomised controlled trial of effect of high-impact exercise on selected risk factors for osteoporotic fractures; HeinonenMS). Osteoporotic fractures among the elderly are common. Higher rates of femoral neck fractures, a weight bearing site, were shown in the training than in the control group. At non-weight bearing sites, such as the distal radius, there was no significant difference between the training and control groups. In the training group there was a significant improvement in vertical jump and predicted oxygen consumption per minute at maximum exercise compared with controls. The results show that high-impact exercises load bones with a rapidly rising force profile, and improve skeletal integrity, muscular performance, and dynamic balance in premenopausal women. If done on a regular basis, this type of exercise may help decrease the risk of osteoporotic fractures in later life. Algorithm-based training can tailor the type of exercise to the subject for a continuous improvement of bone structure. Similarly, (Int J Sports Med 1993; 14(6): 347-352; Exercise-Induced Stress Injuries to the Femur; D. B. Clement) 71 athletes with 74 stress injuries to the femur were studied. Running was the most common activity at the time of injury. Thirty percent of the runners had increased their training duration immediately prior to their first symptom. The mean time to diagnosis and recovery were 6.6 and 10.4 weeks respectively. Substitution of cycling and water exercise for running were the most common therapeutic interventions. There is still a need in the art for subject-tailored methods that can help prevent such fractures and strengthen the bony skeleton.

g. Exercise in patients with heart failure (HF): The American Heart Association Recommendations for Physical Activity in Adults state that being physically active can prevent heart disease and stroke. To improve overall cardiovascular health, they suggest at least 150 minutes per week of moderate exercise or 75 minutes per week of vigorous exercise (or a combination of moderate and vigorous activity). Thirty minutes a day, five times a week is an easy goal to remember. Benefits were shown also when time was divided into two or three segments of 10 to 15 minutes per day. For people who would benefit from lowering their blood pressure or cholesterol, they recommend 40 minutes of aerobic exercise of moderate to vigorous intensity three to four times a week to lower the risk for heart attack and stroke (Clio Med Insights Cardiol 2015; 9:1-9. Clinical Utility of Exercise Training in Heart Failure with Reduced and Preserved Ejection Fraction; Asrar Ul Haq M et al). While itis declared that any recommendation for training in HF should be based on the particular pathology of the patient, the individual's response to exercise, including heart rate, blood pressure, clinical symptoms, and perceived exertion, and on measurements obtained during cardiopulmonary exercise testing, there is no available method to answer that need. Additionally, patient's individual status, including current medications, risk factor profile, behavioral characteristics, personal goals, and exercise preferences, should be taken into consideration. None of the current recommendations provide a solution to that recommendation (The Adv Chronic Dis 2013 May; 4(3): 105-117. Exercise training in chronic heart failure; De Maeyer C et al). It is essential to tailor the prescribed exercise regimen, so that both regimen efficiency and subject safety are guaranteed. There is therefore a need for subject-tailored methods, which will provide a better, more effective, and safer regimen for the continuous improvement of cardiovascular function.

h. Exercise in patients with Alzheimer's disease (AD): Brain function is associated with physical activity. The best exercise regimen for AD patients has yet to be defined. There is increasing evidence that multicomponent training, involving aerobic, muscle strength, and power and balance/coordination exercises, provides important health benefits in AD. (Rachel Pizzie, Alzheimer Dis Assoc Disord. A 2014 January-March; 28(1): 50-57.) Increased physical activity may protect against cognitive decline in AD. Patients with higher physical activity levels performed better on tests of episodic memory and visuospatial functioning. Over subsequent follow-up visits, higher physical activity was associated with small performance gains on executive functioning and working memory tasks in participants with one or more copy of the apolipoprotein ε4 allele (APOE4). In APOE4 non-carriers, slopes of cognitive performance over time were not related to baseline physical activity. These results suggest that cognitively normal older adults who report higher levels of physical activity may have better cognitive performance, but the potential cognitive benefits of higher levels of physical activity over time may be most evident in individuals at genetic risk for AD. Thus, there is a need for subject-tailored methods that can improve adherence and compliance which are important challenges in these populations. When specifying the type, intensity, and overall volume of exercises that should be practiced in order to prevent the development of AD and PD, both subject, disease, type of training, and environment-related factors should be considered.

i. Regular nutritional habits support the maintenance of chronic disease state: overweight and obese (BMI>25 kg/m²) contribute to an inflammatory state of the body and facilitate several disorders, i.e.: metabolic syndrome. Favorable changes in nutritional habits, though initially producing positive outcomes on health, reach a plateau, Ghrelin levels rise and BMI increases and often results in higher levels than before the change in habit was made (Peptides. 2013 November; 49: 138-144. Ghrelin and peptide YY increase with weight loss during a 12-month intervention to reduce dietary energy density in obese women. Brenna R. Hill et al). There is therefore a need to help adjust nutritional habits per subject, in a fashion that would prevent adaptation to and hindering of the health consequences.

### SUMMARY

The following embodiments and aspects thereof are described and illustrated in conjunction with systems, tools and methods, which are meant to be exemplary and illustrative, not limiting in scope. In various embodiments, one or more of the above-described problems have been reduced or eliminated, while other embodiments are directed to other advantages or improvements.

The embodiments show that a personalized-based training regimen of irregular challenged-exercise/training/education/teaching/nutrition, or any maneuver, procedure, or stimuli regimen which is subject-specific and/or organ and/or disease and/or aim of training-tailored can continuously improve response rate and/or prevent or treat organ adaptation, thus improving the effect of training/nutrition regimens, enabling achieving a better effect within a shorter period of time, and with better subject adherence, and reduced training-related complications. In accordance with some embodiments, there is provided herein algorithm-based methods that are subject-specific, organ performance-specific, genetic and phenotypic-specific, and/or physiological target-specific and/or any combination thereof.

In accordance with some embodiments, there are provides herein method(s) and/or device(s)/system(s) implying the method(s), for overcoming organ adaptation for any type of exercise, training, education, teaching-programs, or to device-generated maneuvers, by using a subject-specific, regimen-tailored algorithm. According to some embodiments, since organ adaptation varies among different individuals, each subject is assigned a specific algorithm to overcome it. Similarly, any type of exercise, training, education, learning programs, any chronic disease, requires a tailored algorithm. A subject-tailored continuously or semi-continuously developing randomization-based algorithm for improving organ function is provided in accordance with some embodiments.

In accordance with some embodiments, there are provides herein subject-tailored, continuously or semi-continuously developing, closed loop method(s) and/or device(s)/system(s) implementing randomization-based algorithms for improving organ function is needed. These methods provide, in accordance with some embodiments, training, exercising, teaching or learning regimens that replace the “one for all regular and repetitive” programs. Moreover, they require the brain-muscle-heart connection to be active, which further enhances the beneficial effects of training.

In accordance with some embodiments, there are provides herein subject-tailored, continuously or semi-continuously developing, closed loop method(s) and/or device(s)/system(s) implementing randomization-based algorithms for mitigating or preventing epithelial damage reaching the “asthma” level in professional athletes.

In accordance with some embodiments, there are provides herein subject-tailored, continuously or semi-continuously developing, closed loop method(s) and/or device(s)/system(s) implementing randomization-based algorithms for mitigating or preventing fractures and/or strengthen the bony skeleton.

In accordance with some embodiments, there are provides herein subject-tailored, continuously or semi-continuously developing, closed loop method(s) and/or device(s)/system(s) implementing randomization-based algorithms for improving cardiovascular function.

In accordance with some embodiments, there are provides herein subject-tailored, continuously or semi-continuously developing, closed loop method(s) and/or device(s)/system(s) implementing randomization-based algorithms for quantitatively and qualitatively comparing the effects of different types of brain and physical exercises on well-defined outcomes in AD and/or Parkinson's (PD) populations. In accordance with some embodiments, these algorithms that improve adherence and compliance which are important challenges in these populations.

In accordance with some embodiments, there are provides herein subject-tailored, continuously or semi-continuously developing, closed loop method(s) and/or device(s)/system(s) implementing randomization-based algorithms for adjusting nutritional habits per subject, in a fashion that would prevent adaptation to and hindering of the health consequences.

According to some embodiments, the algorithm disclosed herein, for example, for the applications described hereinabove, may be based on deep machine-learning that benefit by learning from large numbers of subjects with the same disease and the same treatment, as well as enable tailoring of challenged-exercises/training/teaching/education/nutrition regimens to be more beneficial for certain subjects. According to some embodiments, a cell phone-based application, or any other mode of alert system, will send an alert to the subject or to the exercise device, along with the new algorithm to be used with the training. These include for example, but not limited to: treadmills, bicycles, muscle building devices, fitness devices, ellipticals, rowers, roman chair, stair master, any type of gym device, gym machine, gym equipment, using simulators for education, pilot and driving simulators, teaching devices such as simulators for learning languages, or mathematics, or any maneuver or device which can be used for training, exercising, or education of the organ/organs aimed at improving its/their function.

According to some embodiments, there are provided herein devices, systems and methods for generation of exercising, training, education, learning, nutrition and/or or teaching, and/or treatment algorithms in a way which is subject-tailored, organ-function-tailored, topic-tailored, disease-tailored, aim of exercise and/or learning-tailored, closed loop continuously or semi continuously learning for prevention and/or overcoming of adaptation to exercise, education, training or teaching programs, and/or for overcoming partial or complete loss of effect, or non-responsiveness to these regimens, via altering any of the parameters associated with the challenged-exercise/training/teaching/education or maneuvers, by any type of a change which is of relevance for improving the long term effect of said training, or for continuously achieving a better level of performance of an organ or organs. According to some embodiments, this may also be achieved by using devices which generate any type of maneuvers or stimulations. The training algorithms, and/or the algorithm-based devices may be used for continuous prevention or overcoming of adaptation or loss of a maximal effect of all types of training regimens, education sessions.

According to some embodiments, the scope of this disclosure cover any procedure involving organ-related treatment/maneuver/training/teaching/exercise, wherein the procedure parameters are updated within or between the training session periods, for personalizing the algorithm parameters, for increasing the accuracy and/or efficacy of the maneuver, for continuously achieving a better physiological goal/better organ function, for preventing (e.g., continuously preventing) adaptation, and/or for ensuring prolonged maximal effect of training sessions on target organ or on any type of organ performance.

According to some embodiments, the algorithm which provides new challenged-exercise/training/teaching/education regimens is subject-specific, and/or organ function-specific, and/or topic-specific, and/or disease-tailored, and/or maneuver-tailored and/or aim of exercise or teaching-tailored, which is based on alteration of the training regimen, by providing a specific regimen to the organ and/or to any organ in the body using an algorithm-based alteration of training regimens and/or algorithm-based alteration of device-based maneuvers.

According to some embodiments, the algorithm, which provides a new exercise/teaching/education/training regimen is subject-tailored, and/or organ function tailored, and/or topic-tailored, disease-tailored, and/or maneuver-tailored and/or aim of program-tailored, is based on alteration of the regimen, by subject/organ performance/topic/physiological aim-tailored continuously or semi continuously, and includes developing randomization-based algorithms for improving of the organ or organs' function. This method thus overcomes adaptation to any type of monotonic exercise/training/teaching/education/nutritional programs including regimens which involve a gradual increase of level of difficulty, or the use of regular intervals during training sessions.

According to some embodiments, the algorithm enables to exert a positive burden on the brain-target organ connections, for further improving target organ function by enabling the brain to take an active part in the training, thus preventing adaptation and increasing the efficacy of the training by further improving organ performance.

According to some embodiments, the algorithm which provides a new challenged-exercise/training/teaching/learning/education/nutritional regimen is subject-tailored, organ function-tailored, and/or topic-tailored and/or physiological aim-tailored and/or disease tailored, and/or device-generated maneuver-tailored by using a method for continuously or semi continuously improving the ability to reach a better end result, including an ability to reach better endpoints by using a non-gradual, non-stepwise, approach in a subject/organ/topic/environment-tailored approach. This method thus leads to improved organ or organs function within a shorter period of training times, with less adaptation, higher adherence, and less training-associated complications.

According to some embodiments, the subject-tailored continuously developing randomization-based algorithm provides new exercise/training/teaching/learning/education/nutritional regimens which are subject-tailored, and/or organ performance-tailored, and/or topic-tailored and/or physiological aim-tailored and/or disease-tailored, and/or maneuver-tailored by using a method to prevent or ameliorate any type of complication associated with exercise or with training, such as stress fractures, exercise-induced asthma, exacerbation of heart failure, mental problems, lack of adherence to training and others.

According to some embodiments, use of these algorithm-based regimens improves adherence to the exercise/training/teaching/learning/education regimen/program.

According to some embodiments, there are provided herein methods, schemes, protocols and/or regimens for exercise/training/teaching/learning/education/nutritional and/or any type of maneuver administration by devices, which are based on algorithms configured to update parameters within the period of the exercise/training/teaching/learning/education/nutritional and/or any type of maneuver administration by devices, for personalizing thereof, increasing efficacy thereof and overcoming adaptation thereto. According to some embodiments, the algorithms take into consideration any type of treatment/exercise/training/teaching/learning/education program or device-based procedure administration in accordance or discordance with subjects'-dependent factors, background disease dependent factors, circadian rhythm, or any other type of factor, as well as the overall aim of the maneuver used or type of training which affects the response to said regimen directly or indirectly.

According to some embodiments, the parameters are determined and updated using a machine learning system, which provides parameter values based on feature values received from and/or related to the user.

According to some embodiments, an algorithm-based new exercise/training/teaching/learning/education/nutritional regimen is being generated for improving physical or mental activity, for improving the function of an organ or organs, or for treatment of obesity, infectious, metabolic, endocrinology, malignant, immune-mediated, inflammatory condition, inborn error of metabolism, pain, microbiome-related disorders, neurological disease, any type of acute or chronic disease or condition in which improvement of organ performance is desired, and in any type of condition in which circadian rhythm is relevant, including jet lag, and any type of chronic medical problem or health condition which can benefit from improved organ function, or any condition in which a healthy subject wishes to improve the status or function of organs, or wishes to learn any topic, will be irregular aimed at improving response rate and maximizing the effect of the procedure, shortening the time required for reaching a maximal effect, and with less complications.

According to some embodiments, the parameters are determined and updated using a machine learning system, which provides parameter values based on feature values received from and/or related to the user.

According to some embodiments, the machine learning system is a deep learning system, in which the learning on some features is guided learning, while learning on other features is unguided learning.

According to some embodiments, the number of layers/levels of the deep machine learning depends on the number of features or on the number of associations between them.

According to some embodiments, the user updates the machine with inputs indicative of progress towards the targeted physiological goal, and the learning machine provides an updated method of challenged-exercise/training/teaching/learning/education regimen or maneuver administration according to the tailored parameters relevant for the subject/procedure based on data learned from the user and/or other users.

According to some embodiments, as used herein, the term physiological goal or target may refer to value, gradient, or change in physiological measure or parameter in a desired direction, or learning of a new topic, or achieving a better score in a test which reflects improved organ/organ performance. For example, the goal may be avoiding development of adaptation to training aimed at improving the ability to run or study faster. In this case, such a goal may be avoiding tolerance to a certain challenged-exercise/training/teaching/learning/education/nutritional regimen by setting a deep-machine learning closed-loop individual based-algorithm that sets a new regimen for the subject. The new regimen is designed with or without setting a specific range as a target for parameter/value change.

According to some embodiments, a user may update the machine, or the machine may receive inputs from the user and/or other users that are being used to update the algorithm in a way that enables redirecting or further defining the changes in the exercise regimen via providing of a change in one or more of the parameters which are relevant to the challenged-exercise/training/teaching/education regimen and to the subject. The learning machine provides updated parameters based on data being continuously or semi-continuously learned from other users. The data received is continuously or semi-continuously analyzed based on sub groups of subjects, including based on exercise/training/teaching/learning/education/nutritional regimen organ function-related parameters/disease-related parameters, targets to be achieved, subject-related parameters such as age gender, comorbidities, concomitant medications and other factors which are subject and/or disease and/or drug and/or overall aim of regimen-related.

According to some embodiments, there is provided a mobile phone-based system, or any other type of an alert system, for dispensing instructions to subjects, including an update module, computationally configured to receive a plurality of feature values, and provide the relevant parameters for setting a new exercise/training/teaching/learning/education regimen. These parameters may be changes based on type of input received from the subject, including measurements of physiological parameters such as results of tests in the topic, achievements in competitions, pulse, respiratory rate, oxygen consumption, and information received from EEG, ECG, EMG, MRI, CT, PET, PET/CT, US, X-ray, DEXA, blood tests, any type of physiological or pathological biomarkers, parameters which are directly or indirectly related to organ performance and/or to the subject.

According to some embodiments, the processing circuitry of the update module is operated to facilitate machine-learning capabilities, wherein supervised and/or unsupervised learning is utilized.

According to some embodiments, the machine learning capabilities include deep learning capabilities.

According to some embodiments, the physiological goal is avoiding development and/or overcoming adaptation, habituation, or tolerance to long-term training.

According to some embodiments, the machine learning success factor is maintaining physiological change and/or improvement in target organ function.

According to some embodiments, the features of the machine learning are selected from a list including: type of exercise/training/teaching/learning/education/nutritional regimen used, type of maneuver, type of target organ, organ performance/final aim to be achieved by exercise/training/teaching/learning/education/nutritional program, background disease, mode of administration of the regimen, microbiome-associated factors, concomitant medications; and list of subject-related parameters including performance on previous tests, aim of training and teaching, performance of previous competitions, age, weight, gender, ethnicity, geography, pathological history/state, past/present medications, temperature, metabolic rate, glucose levels, blood tests and any physiological or pathological parameters that may be measured whether directly or indirectly associated with the aim of training or with the physiological target; any type of biomarker which is directly or indirectly associated with an exercise/training/teaching/learning/education regimen, and/or disease and/or to the drug and/or with a subject or a subgroup of subjects.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some, or none of the enumerated advantages.

In addition to the exemplary aspects and embodiments described above, further aspects and embodiments will become apparent by reference to the figures and by study of the following detailed descriptions.

According to some embodiments, the parameters are determined and updated using a machine learning system, which provides parameter values based on feature values received from and/or related to the user.

According to some embodiments, the machine learning system is a deep learning system, in which the learning on some features is guided learning, while learning on other features is unguided learning.

According to some embodiments, the number of layers/levels of the deep machine learning depends on number of features or on the number of associations between them.

According to some embodiments, the user updates the machine with inputs indicative of progress towards the target physiological effect goal, and the learning machine provides updated algorithm and/or device-derived parameters based on data learned from the user and/or other users, while a different physiological goal may be given to other users with similar feature values such as organ function, performance on tests, and scores which are of relevance to organ performance, race, age, gender, health conditions, concomitant medications, and so on, as well as data specific to the user.

According to some embodiments, as used herein, the term physiological goal or target may refer to value or degree of organ performance on a test or competition. Once a goal is achieved the type of challenged-exercise/training/teaching/education/nutritional regimen used or different parameters relevant to the regimen may change only to maintain it or to further improve it, or, for example, when the user gets closer to the target value, the challenged-exercise/training/teaching/learning/education regimen may be altered.

According to some embodiments, as used herein, the term physiological goal or target may refer to a gradient or change in a physiological measure/parameter in a desired direction. For example, the goal may be improved mental function in Alzheimer disease, or improved swimming speed, or better performance on a math test, without determining an exact value as a target for the physiological measure/parameter.

According to some embodiments, a user may update the machine, or the machine may receive inputs from the user and/or from other users. These inputs are used to update the algorithm in a way that enables to redirect or further define the ideal parameters of the algorithm-based challenged-exercise/training/teaching/learning/education regimens or of device-generated maneuver regimen and/or defined stimuli being administered to the user following a closed-loop system for achieving the best results possible for the subject.

According to some embodiments, the newly generated training regimen further contributes to progression towards a target physiological goal, and the learning machine provides updated algorithm and/or procedure parameters based on data being continuously or semi continuously learned from the user and/or other users.

According to some embodiments, the data is continuously or semi-continuously received, and is analyzed based on factors associated with the target organ function, the overall aim to be achieved, performance of previous tests, scores achieved in previous competitions, background diseases, type of medications used, nutritional status, status of other organs, and/or subgroups of subjects, targets of physiological levels to be achieved, and biomarkers which are of relevance to the exercise/training/teaching/learning/education regimen, and others.

According to some embodiments, there is provided a system for a closed loop algorithm-generator and/or maneuver-generator and stimulation units, including an update module, computationally configured to continuously or semi continuously receive a plurality of feature values, and provide a new training regimen, and/or device-generated maneuver/stimulation parameters, based on, at least one sensor, configured to measure a physiological or pathological property, and provide a signal indicative thereof, and output device that notifies the subject when and how to perform the challenged-exercise/training/teaching/learning/education/nutritional regimen.

According to some embodiments, the loop will include a maneuver/stimulation device, including: a maneuver/stimulation inducer, configured to generate a challenged-exercise/training/teaching/learning/education/nutritional regimen based on stimulation parameters to affect a physiological or pathological change in a target organ or organs.

According to some embodiments, the system includes a communication unit, configured to allow transfer of data to the main part of the algorithm which set up the output, and/or a signal to a maneuver/stimulation device for modifying one or more of the exercise/training/teaching/learning/education/nutritional regimen parameters and/or maneuver/stimulation parameters, an update module, including a processing circuitry, configured to: obtain a signal from the sensor, determine the algorithm and/or maneuver/stimulation parameters based on the signal obtained from the sensor, provide an alert to the new regimen and/or provide the device with the determined maneuver/stimulation parameters via the communication unit.

According to some embodiments, the processing circuitry of the update module is operated to facilitate machine-learning capabilities, wherein supervised and/or unsupervised learning is utilized.

According to some embodiments, the algorithm is provided for continuously achieving a desired physiological change, and the learning machine success factor is achieving, maintaining, and continuously improving this physiological change.

According to some embodiments, the physiological goal is a continuous prevention of adaptation or partial/complete loss of an effect to challenged-exercise/training/teaching/learning/education/nutritional regimen of any organ during a training regimen in healthy subjects, and for subjects with any type of acute or chronic disease or condition which requires improved organ function, or conditions in which a subject wishes to improve the function of one of their organs, such as when aiming at improving language knowledge, improve running capability, heart function, lung function, muscle function, any type of sport activity, studying of any subject, lowering body weight, managing glucose levels, lowering blood pressure, treating cancer, treating acute or chronic pain, circadian rhythm related disorders including jet lag, treating epilepsy or any neurological disease, any type of mental condition such as Alzheimer disease, treating any metabolic disease, treating endocrinology disorders, treating genetic disorders, treating an inborn error of metabolism, treating microbiome-associated conditions, treating any liver disease, treating all types of diabetes, treating any infectious disease including viral, bacterial, fungal infection, treating inflammatory or immune-mediated disease. For example, such immune-related disorders may be an autoimmune disease, graft rejection pathology, inflammatory bowel disease, nonalcoholic fatty liver disease, hyperlipidemia, atherosclerosis, metabolic syndrome or any of the conditions including the same.

Examples of autoimmune disorders include, but are not limited to: Alopecia Areata, Lupus, Ankylosing Spondylitis, Meniere's Disease, Antiphospholipid Syndrome, Mixed Connective Tissue Disease, Autoimmune Addison's Disease, Multiple Sclerosis, Autoimmune Hemolytic Anemia, Myasthenia Gravis, Autoimmune Hepatitis, Pemphigus Vulgaris, Behcet's Disease, Pernicious Anemia, Bullous Pemphigoid, Polyarthritis Nodosa, Cardiomyopathy, Polychondritis, Celiac Sprue-Dermatitis, Polyglandular Syndromes, Chronic Fatigue Syndrome (CFIDS), Polymyalgia Rheumatica, Chronic Inflammatory Demyelinating, Polymyositis and Dermatomyositis, Chronic Inflammatory Polyneuropathy, Primary Agammaglobulinemia, Churg-Strauss Syndrome, Primary Biliary Cirrhosis, Cicatricial Pemphigoid, Psoriasis, CREST Syndrome, Raynaud's Phenomenon, Cold Agglutinin Disease, Reiter's Syndrome, Crohn's Disease, Rheumatic Fever, Discoid Lupus, Rheumatoid Arthritis, Essential Mixed, Cryoglobulinemia Sarcoidosis, Fibromyalgia, Scleroderma, Grave's Disease, Sjogren's Syndrome, Guillain-Barre, Stiff-Man Syndrome, Hashimoto's Thyroiditis, Takayasu's Arteritis, Idiopathic Pulmonary Fibrosis, Temporal Arteritis/Giant Cell Arteritis, Idiopathic Thrombocytopenia Purpura (ITP), Ulcerative Colitis, IgA Nephropathy, Uveitis, Insulin Dependent Diabetes (Type I), Vasculitis, Lichen Planus, and Vitiligo. Graft versus host disease (GVHD), or to prevent allograft rejection. According to some embodiments, an autoimmune disease treated by the methods, devices and/or systems disclosed herein may be any one of rheumatoid arthritis, atherosclerosis, asthma, acute and chronic graft versus host disease, systemic lupus erythmatosus, scleroderma, multiple sclerosis, inflammatory bowel disease, psoriasis, uvietis, thyroiditis and immune mediated hepatitis. The methods disclosed herein may be applicable for the treatment of hypertension, diabetes, and the metabolic syndrome, abdominal obesity Atherogenic dyslipidemia, elevated blood pressure, insulin resistance or glucose intolerance, prothrombotic state, and proinflammatory state (e.g., elevated C-reactive protein in the blood). People with the metabolic syndrome are at increased risk of coronary heart disease and other diseases related to plaque buildups in artery walls (e.g., stroke and peripheral vascular disease) and type 2 diabetes.

The methods disclosed herein may be applicable for the treatment of cancer (malignancy). Malignancy may be selected from the group consisting of: carcinomas, melanomas, lymphomas, myeloma, leukemia and sarcomas. Malignancies may include, but are not limited to: hematological malignancies (including leukemia, lymphoma and myeloproliferative disorders), hypoplastic and aplastic anemia (both virally induced and idiopathic), myelodysplastic syndromes, all types of paraneoplastic syndromes (both immune mediated and idiopathic) and solid tumors (including lung, liver, breast, colon, prostate GI tract, pancreas and Kaposi). More particularly, the malignant disorder may be hepaotcellular carcinoma, colon cancer, melanoma, myeloma, acute or chronic leukemia.

The above method may be applicable for any maneuver or exercise or nutrition relevant for treatment of neurological or mental disorders, and pain, as well as for any type of inborn error of metabolism; Peripheral or central neurological disorders: Huntington diseases; ALS; Dementia; Alzheimer's disease; treatment of genetic diseases; treatment of any endocrine disorder.

For example, sport and teaching activities include running, bicycles, muscle building devices, fitness devices, elliptical, rowers, roman chair, stair master, trampoline, pole, any type of gym device or gym machine or gym equipment, using simulators for education, pilot and driving simulators, teaching devices such as simulators for learning languages, software used for education and mathematics, or any maneuver or device which may be used for training, exercising, or education of the organ or organs aimed at improving its/their function or altering/changing nutritional habits.

According to some embodiments, the machine learning capabilities include deep learning capabilities.

According to some embodiments, the features of the machine learning are selected from a list including: target organ function-associated factors, aim of exercise/training/teaching/learning/education/nutritional regimens-associated factors, disease-associated factors, other organs-associated factors, drug-related factors, and/or subject-associated factors such as performance of previous tests, scores achieved in competitions, accomplishments on tests relevant for organ performance, age, weight, periodic caloric intake and output, gender, ethnicity, geography, pathological history/state, temperature, metabolic rate, glucose levels, blood tests and any physiological or pathological parameters and/or biomarkers that may be measured whether directly or indirectly related with the physiological target and with the desired performance goal.

According to some embodiments, the output of the algorithm may be in a form of a notification being delivered to the subject via a cell phone-based application, or by any other alert method, that instructs or alarms the subject on the regiment to be used and/or change in parameters which are relevant to the exercise including during the session itself in real time or not.

According to some embodiments, the algorithm and/or maneuver/stimulation inducer is configured to affect a challenged-exercise/training/teaching/learning/education/nutritional regimens and/or a maneuver or procedure and/or any type of device, including medical device, by providing any type of measures or parameters relevant to the target organ performance whose function the subject wishes to improve, including procedures or devices that provide physical or mental exercises, or devices which use any type of maneuver/stimulation including magnetic, mechanical, electrical, temperature-based, ultrasound-based, or any other type of signal to the target body part, by physical movement, using numerous levels of trainings, various degrees of difficulties of the exercise, types or rate and rhythms of the maneuver/stimuli with various frequencies, amplitudes, durations, and interval, in a structured or random manner, or other types of direct or indirect stimuli.

According to some embodiments, the algorithm provides a method for a constant prevention of adaptation to challenged-training which precludes achieving the maximal effect the subject may achieve from the challenged-exercise/training/teaching/learning/education/nutritional regimens, or partial loss of effect of the regimen, or partial/complete non-responsiveness to training, or reaching a plateau from which no further improvement is gained, by setting up a continuous irregularity within a specific said range that is predetermined for each subject, and for each organ that it being targeted, and for each type of exercise/training/teaching/learning/education regimen and/or device-generated maneuver being used.

According to some embodiments, the algorithm provides a method for prevention of adaptation to exercise/training/teaching/learning/education/nutritional regimen, or loss of effect of training, or non-responsiveness to training, by continuously setting up a new regimen and/or a new device-related signal, with an irregularity within a specific said range that will be predetermined for each type of training regimen and for each subject.

According to some embodiments, the sensor is configured to measure, mental function, any type of physical activity, any score on a test, any performance on a competition, temperature, oxygen levels, blood pressure, and/or blood tests, organ activity, and/or any physiological or pathological parameters or biomarkers, that may be measured whether directly or indirectly-associated with the physiological target of organ performance.

According to some embodiments, there is provided a challenged-exercise/training/teaching/learning/education/nutritional regimen and/or a device which is aimed at targeting said organ, inducing devices which provide stimulation for brain, or abdominal stimulation, or any organ stimulation, whether this organ is associated with the target of the regimens, or with the target organ for the training or not, including a maneuver/stimulation device inducer, configured to generate a stimulation action based on parameters that affect a physiological change in an organ or organs, and a communication unit, configured to allow transfer of data between the device and an update module, wherein the update module includes a processing circuitry, configured to obtain a signal from at least one sensor indicative of a physiological or pathological property, determine algorithm and/or maneuver/stimulation parameters based on the signal obtained from the sensor, and provide the device with the determined algorithm/maneuver parameters via the communication unit.

According to some embodiments, a method for a continuous/semi-continuous/non-continuous/conditional, closed-loop for any organ maneuver/stimulation or modification, including providing/placing in the proximity of a target body part a modification/stimulation-device, or any device which may provide any type of a signal, or may induce any direct or indirect change in organ function, or transplanting a maneuver/stimulation-device, with a maneuver/stimulation inducer, providing initial parameters to the device, based on initial acquired information and on a desired physiological change in organ function, providing maneuver/stimulation via the inducer, or providing any type of signal or effect that may alter organ function, based on initial stimulation parameters, obtaining information from the user and/or device or other sources, and updating the relevant exercise and procedure parameters based on the obtained information.

According to some embodiments, a method for a continuous, semi-continuous, conditional, or non-continuous closed loop for generating a new exercise/training/teaching/learning/education regimens by providing an alert for the specific algorithm-dependent parameters, time, mode of administration, degree of difficulty, or any other training-related parameter.

According to some embodiments, continuously updating the newly-generated challenged-exercise/training/teaching/learning/education/nutritional regimen based on alteration of any of the regimen relevant parameters, and/or training device associated parameters, including for example: degrees of difficulties, times for performance of the exercise, combining several types of exercises or procedures, and/or using stimulation parameters, includes utilizing machine learning capabilities. According to some embodiments, the machine learning capabilities include deep learning in a closed loop method.

According to some embodiments, the machine learning capabilities are configured to be operated on a set of features by receiving values thereof. According to some embodiments, the output new regimen is provided to the subject by a cell-phone or computer-based alert system, or any other type of an alert system and/or by a maneuver/stimulation-device including a wearable/implantable device. According to some embodiments, the stimulation device or the device that affects organ function is configured to be swallowed by a user. According to some embodiments, the device is configured to be placed on the body of the user, or to be used via any device which is in direct or indirect contact with the human body or with the target organ.

According to some embodiments, a physiological goal is an improvement in target organ function, improved learning capability, or disease conditions or improving health by prevention of adaptation or loss of an effect or non-responsiveness to challenged-exercise/training/teaching/learning/education/nutritional regimens and/or improving the performance of any organ or organs.

According to some embodiments, there is provided herein, a computer implemented method for improving target cells, tissue, organ and/or whole body function and/or performance by preventing or mitigating adaptation to at least one regimen selected from treatment, maneuver, challenged-exercise, training, learning, and nutritional regimens, the method includes: receiving a plurality of physiological and/or pathological parameters related to the subject; applying a closed loop machine learning algorithm to the plurality of physiological and/or pathological parameters; determining subject-specific output parameters relating to the at least one of the treatment, challenged-exercise, training, learning and nutritional regimens, wherein the subject-specific output parameters facilitate preventing or mitigating cell, tissue and/or organ adaptation to the at least one regimen; and utilizing the subject-specific output parameters to improve the machine learning algorithm by applying a subject-tailored continuously or semi continuously randomization-based or non-randomization based algorithm thereby facilitating continual improvement of cell, tissue and/or organ function and/or performance.

According to some embodiments, preventing or mitigating adaptation may include overcoming partial or complete loss of effect of (or non-responsiveness to) the at least one regimen.

According to some embodiments, the method may further include utilizing a subject-tailored continuously or semi continuously algorithm which is uses a randomization-based and/or non-randomization based algorithms configured to use or combine one or more algorithm training tasks whether directly related or not directly related to the target cells, tissue, organ and/or body for improving function and/or performance thereof.

According to some embodiments, the method may further include, utilizing a stimulation device, providing to the subject stimulation for maximizing the effect of the at least one regimen.

According to some embodiments, the method may further include updating at least one of the subject-specific output parameters. According to some embodiments, updating may include updating amplitude, frequency, interval, duration of the at least one of the subject-specific output parameters or any combination thereof. According to some embodiments, the method may further include updating may include updating amplitude, frequency, interval, duration of the at least one of the subject-specific output parameters or any combination thereof.

According to some embodiments, the method may further include determining stimulation parameters.

According to some embodiments, the output parameters may be updated based on data being continuously or semi continuously collected from the subject.

According to some embodiments, the machine learning algorithm further considers personal data selected from a group consisting of: subject performance, cell/tissue/organ function-related scores, parameters relevant to cell/tissue/organ performance, age, weight, waist circumference, target organ, and other organs' function, caloric intake and output, gender, ethnicity, geography, pathological history/state, temperature, metabolic rate, brain function, health status, heart, lung muscle function, blood tests, and any physiological or pathological biomarkers, a subject's health related parameter or any combination thereof.

According to some embodiments, the at least one of the physiological and/or pathological parameters is obtained from a sensor.

According to some embodiments, the method may further include notifying the subject, in real time, of recommended regimen related parameters or changes thereof.

According to some embodiments, the method may further include utilizing an external, wearable, swallowed and/or implanted device for evoking a reaction in the target cells, tissue and/or organ for continually improving function and/or performance thereof.

According to some embodiments, the method may further include administering challenged-exercise regimen, training regimen, education regimen, nutritional regimen or device-generated maneuvers regimens to the subject.

According to some embodiments, the method may further include updating the challenged-exercise/training/teaching/learning/playing/education regimens/nutritional regimens, and/or device generated maneuvers or stimulation parameters, wherein updating includes utilizing machine-learning capabilities. According to some embodiments, the machine learning capabilities include closed-loop deep learning. According to some embodiments, the machine learning capabilities are configured to be operated on a set of features by receiving values thereof.

According to some embodiments, the method may be used for improving organ function in healthy subjects who wish to improve muscle, heart, lung, skin, brain on any other tissue/organ/organs performance, and/or for improving training capabilities of any tissue/organ/organs, improving education, or teaching, and/or for treatment of obesity, infectious, metabolic, endocrinology, malignant, immune-mediated, inflammatory condition, inborn error of metabolism, pain, microbiome-related disorders, neurological disease, fibrosis in an organ, desynchronosis or circadian dysrhythmia.

According to some embodiments, the treatment may include a drug treatment, a device treatment or a combination thereof.

According to some embodiments, there is provided herein, a system for improving target cells, tissue and/or organ function and/or performance by preventing or mitigating adaptation to at least one regimen selected from treatment, challenged-exercise, training, learning, and nutritional regimens, the system includes a processor configured to: receive a plurality of physiological and/or pathological parameters related to the subject; apply a closed loop machine learning algorithm to the plurality of physiological and/or pathological parameters; determine subject-specific output parameters relating to the at least one of the treatment, challenged-exercise, training, learning and nutritional regimens, wherein the subject-specific output parameters facilitate preventing or mitigating cell, tissue and/or organ adaptation to the at least one regimen; and utilize the subject-specific output parameters to improve the machine learning algorithm by applying a subject-tailored continuously or semi continuously randomization-based or non-randomization based algorithm thereby facilitating continual improvement of cell, tissue and/or organ function and/or performance.

According to some embodiments, preventing or mitigating adaptation may include overcoming partial or complete loss of effect of (or non-responsiveness to) the at least one regimen.

According to some embodiments, the processor is further configured to utilize a subject-tailored continuously or semi continuously ongoing developed randomization-based or non-randomization based algorithm configured to combine one or more algorithm training tasks whether directly related or not directly related to the target cells, tissue, organ and/or whole body for improving function and/or performance thereof.

According to some embodiments, the system may further include a stimulation device configured to provide to the subject stimulation for maximizing the effect of the at least one regimen. The stimulation device may be configured to provide signal to a target body part, by physical movement, mechanical signal, electric signal, electromagnetic signal, sonic signal, ultrasound signal, temperature alteration or any combination thereof.

According to some embodiments, the processor may further be configured to update at least one of the subject-specific output parameters. According to some embodiments, updating may include updating amplitude, frequency, interval, duration of the at least one of the subject-specific output parameters or any combination thereof.

According to some embodiments, the processor is further configured to determine stimulation parameters.

According to some embodiments, the processor is further configured to update the output parameters based on data being continuously or semi continuously collected from the subject.

According to some embodiments, the machine learning algorithm further considers personal data selected from a group consisting of: subject performance, cell/tissue/organ function-related scores, parameters relevant to cell/tissue/organ performance, age, weight, waist circumference, target organ, and other organs' function, caloric intake and output, gender, ethnicity, geography, pathological history/state, temperature, metabolic rate, brain function, health status, heart, lung muscle function, blood tests, and any physiological or pathological biomarkers, a subject's health related parameter or any combination thereof.

According to some embodiments, the system may further include a sensor configured to provide signals indicative of the at least one of the physiological and/or pathological parameters.

According to some embodiments, the system may further include an output module configured to notify the subject, in real time, of recommended regimen related parameters or changes thereof.

According to some embodiments, the system may further include an external, wearable, swallowed and/or implanted device configured to evoke a reaction in the target cells, tissue and/or organ for continually improving function and/or performance thereof.

According to some embodiments, the processor is further configured to recommend/update a challenged-exercise/training/teaching/learning/playing/education regimens/nutritional regimens, and/or device generated maneuvers or stimulation parameters, wherein updating includes utilizing machine-learning capabilities. The machine learning capabilities may include closed-loop deep learning capabilities. The machine learning capabilities may be configured to be operated on a set of features by receiving values thereof.

The machine learning capabilities the treatment may include a drug treatment, a device treatment or a combination thereof.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

The non-obviousness of some of these embodiments comes from the fact the claims are made to use an algorithm which is subject-tailored and/or disease and/or exercise-tailored, and/or performance-target to be reached in the organ function-tailored, by a way of altering the training/nutritional-relevant parameters and/or time and/or method of exercise administration and/or combination of different exercises, and/or combination of different degrees of difficulties and/or combination of exercises which target associated organs and/or use of maneuver/stimulation to any organ and/or by using devices and/or any type of physical or mental exercise as an adjuvant to the main exercise, for improving the function of the organ or organs, for improving overall capability of an organ or subject, for prevention/treatment of adaptation to training/nutrition, or as sole treatment for acute and/or chronic diseases, are not expected based on the current knowledge of organ function, physical or mental exercises, and chronic therapies/regimens.

### BRIEF DESCRIPTION OF THE DRAWINGS

Examples illustrative of embodiments are described below with reference to figures attached hereto. In the figures, identical structures, elements or parts that appear in more than one figure are generally labeled with the same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1 schematically illustrates a functional block diagram of a system which accumulates subject-related, performance-related, device-related, and/or target organ-related parameters according to some embodiments and based on the use of a predetermined range for each challenged-exercise/training/teaching/learning/education regimen.

FIG. 2 schematically illustrates a functional block diagram of the closed loop-based algorithm for improving the challenged-exercise/training/teaching/learning/education regimen to continuously prevent target organ adaptation and to improve target organ function, and/or loss of response to a regimen, and improving performance, according to some embodiments.

FIG. 3 schematically illustrates a flow chart of a method for providing updated challenged-exercise/training/teaching/learning/education regimen using said program, procedure, maneuver, or device which can improve organ function, and/or combination of maneuvers, procedures, drugs, devices, and/or medical device, and/or stimulation pattern using device or procedure or method or software which can improve organ function, according to some embodiments.

FIG. 4 schematically illustrates a method for providing a new exercise regimen to a person running on a treadmill with an external muscle stimulation to leg muscles for improving running capability and for continuous prevention of adaptation or loss of a chronic effect by using a regular exercising program.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

According to some embodiments, there are provided herein algorithms, methods, devices, and systems for improving organ function and overall performance, by continuously providing a new challenged-exercise/training/teaching/learning/education/nutritional regimen, and for preventing, mitigating or treating partial/complete loss of effect of training programs due to adaptation, used by a subject in a need thereof, and for continuously maximizing the effect of the exercise/training/teaching/learning/education regimens, on function of a target organ, the method being continuous/semi-continuous/conditional/or non-continuous closed loop deep learning individualized molecular/cellular/tissue or any other organ stimulation.

According to some embodiments, there are provided herein devices, systems and methods for altering the parameters relevant to the exercise/training/teaching/learning/education/nutritional regimen and/or combining different exercise regimens and/or using target organ maneuver/stimulation or any procedure or any device which can assist, and for improving the sustainability and continuously improved organ function.

According to some embodiments, any organ stimulation, wherein the output and device parameters are updated within the exercise/training/teaching/learning/education/nutritional period, for personalizing the procedure, training, or device parameters, and increasing accuracy and efficacy of the output procedure regimen and/or the stimulation or any other type of procedure provided by the device, or any type of treatment, for achieving the desired physiological goal and to prevent long-term adaptation, for ensuing prolonged effect and improving the function of the organ or physiological pathway.

According to some embodiments any type of any output exercise/training/teaching/learning/education/nutritional regimen and/or organ maneuver/stimulation or any signal provided by a device, wherein the stimulation or other parameters are updated within the training period, for personalizing the stimulation or other signals and characteristics to increase the accuracy and efficacy of the regimen for achieving the desired physiological goal and improving the overall function of the organ.

According to some embodiments, the parameters are determined and updated using a machine learning system, which provides parameter values based on feature values received from and/or related to the user and to their performances.

According to some embodiments, the machine learning system may be a deep learning system, in which the learning on some features is guided or supervised learning, while learning on other features is unguided or unsupervised learning.

According to some embodiments, the number of layers/levels of the deep machine learning depends on the number of features.

According to some embodiments, the user updates the machine with progress towards the target physiological goal, for an overall improvement of organ performance, and the learning machine provides updated challenged-exercise/training/teaching/learning/education/nutritional regimen-relevant parameters and regimens, and/or stimulation or other device-related parameters based on data learned from the target organ function, and/or subject performance, and/or disease, and/or medications, and/or a subject or subgroup of subjects, and/or type of exercise/training/teaching/learning/education/nutritional regimen-related or non-related biomarkers or parameters, or combinations of regimens and/or user and/or other users, that may be given to other users with similar feature values such as performance, scores related to the target function, race, age, gender, health conditions and so on, as well as data specific to the user, for example progress towards target running speed under predetermined conditions and the like.

According to some embodiments, user inputs may include any type of physiological or pathological parameters, as well as personal and environmental parameters which are relevant directly or indirectly to the exercise/training/teaching/learning/education/nutritional regimens or procedures. These parameters may be of relevance to a subject, or to a specific regimen, or to a specific organ and not necessarily to all subjects.

According to some embodiments, the user may update the machine or the machine may receive inputs from the user and/or from other users which are being used to update the algorithm in a way that enable to redirect or further define the exercise/training/teaching/learning/education/nutritional regimens and/or the maneuver/stimuli or other signals administered by a device to the user following a closed-loop system.

The learning machine provides updated exercise/training/teaching/learning/education/nutritional regimens and/or maneuver/stimulation or other device-based procedures, parameters based on data being continuously or semi continuously learned from the user and/or other users. The data received in real time or not, is continuously analyzed based on subgroups of subjects, organ function parameters, and biomarkers which are directly and indirectly associated with the organ, the subject, subject performance, scores achieved in tests, background disease, age, gender, concomitant diseases, concomitant medications, any type of exercise/training/teaching/learning/education/nutritional regimen, related or non-related biomarkers, caloric intake, physical activity, and others.

As used herein, the terms “learning machine”, “update module” and “update system” are interchangeably used, and refer to an integrated or communicatively linked component of the system, which is configured to receive input data in form of user data (such as parameter directly or indirectly associated with the function of the organ, weight, medical state, gender age and the like), features which measure directly or indirectly relevant bodily indications that generate based thereon maneuvers/stimulations or other device-generated procedures-parameters, a set of exercise/training/teaching/learning/education/nutritional-related parameters, thus forming a new exercise/training/teaching/learning/education/nutritional regimen and/or a new device-generated stimulation or procedure plan based on current inputs, historic inputs and/or preconfigured data from the user, multiple users and/or models of users.

According to some embodiments, the input data on the user along with the input received from other users on a continuous or semi continuous basis is being processed by the controller, which is based on a closed loop system that continuously evaluates the distance of the tested parameter from the level to be achieved or the direction and/or rate of changes in the physiological or pathological measurement/parameter, generates an improved algorithm being transformed into new output.

According to some embodiments the algorithm provides a method for continuously improving organ function and for prevention of long term adaptation, and prevention of loss of an effect to a regular perpetual exercise regimen, by setting up an irregularity within a specific said range that is be pre-determined for each exercise or procedure, or for combination of several types of exercises based on their pattern of efficacy, and based on performance output received from the organ or the subject.

According to some embodiments the algorithm provides a method for improving organ function and for prevention of long term adaptation, and prevention of loss of an effect to a regular exercise/training/teaching/learning/education/nutritional regimen, by setting up an irregularity in the mode of a challenged exercise/training/teaching/learning/education/nutritional regimen administration, irregularity in the combination of various exercises and procedures, or any type of irregularity relevant to the regimen, or to device-generated procedures or stimulation or therapies.

The output may be in a form of an alert delivered to the subject via a cell phone-based application, or by any other method, which will instruct the subject on the exercise/training/teaching/learning/education/nutritional regimen or parameters which are relevant to the exercise or maneuver.

According to some embodiments, the output can be delivered by device generated procedure or stimulation inducer is configured to affect a maneuver/stimulation by providing a mechanical, magnetic, electrical, temperature-based, ultrasound based, or any other type of a signal or other maneuver generated by the device to the target body part or any other body part, by physical movement, using various types of rate and rhythms of stimuli with various frequencies, amplitudes, durations, and interval, in structured or random manner, or other types of direct or indirect stimuli.

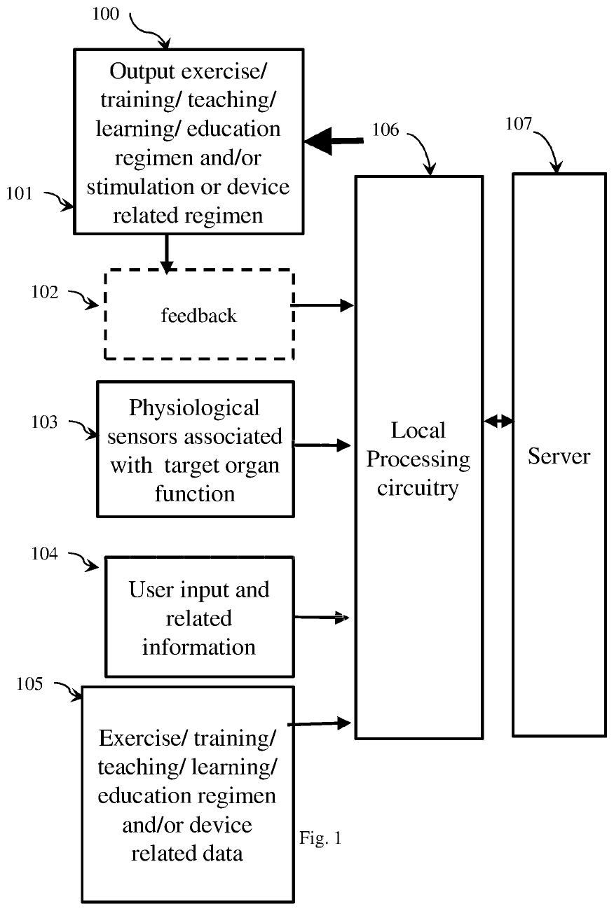

Reference is now made to FIG. 1 of an output exercise alert device and/or maneuver/stimulation system 100, according to some embodiments. According to some embodiments, system 100 includes a challenged-exercise/training/teaching/learning/education regimen alert output device and/or maneuver/stimulator 101, which is configured to provide exercise/training/teaching/learning/education regimen alert output and/or device-generated procedure or stimulation to a target body part (abdomen, brain, or any other organ in the body), to achieve a desired physiological effect for improving of organ function, optionally one feedback mechanism 102 associated with training output and/or stimulator 101, configured to provide measurements of physiological indicators relevant to target organ function, or any other disease related or non-related biomarker, or alternatively, technical information related to 101, such as battery charge level. These parameters may be related or indirectly related to the physiological target which the algorithm is aimed at improving.

According to some embodiments, system 100 further includes additional external sensors 103, for example pulse, rate of breathing, oxygen saturation, blood tests that provide data on the target organ function or on overall body function, or any other test and the like, or any type of performance of the subject, which along with the information from feedback mechanism 102 are provided to a local processing circuitry 102 which is configured to control the operation of 101 based on inputs that include measurements of external or internal sensors 103, and optional feedback mechanism 102. According to some embodiments, processing circuitry 106 is further configured to obtain inputs of user related information 104 and other user inputs including exercise/training/teaching/learning/education regimen and/or device related data 105, based on which, the algorithm and/or device output parameters are determined.

According to some embodiments, external sensors 103, may be organ or exercise/training/teaching/learning/education regimen-related biomarker sensors, configured to provide local processing circuitry 106 with information indicative of the target organ function and exercise/training/teaching/learning/education regimen-target parameters of the user at certain times. According to some embodiments, a user may be instructed or advised to measure their organ function and/or regimen and/or disease-associated biomarker periodically, or any other parameter that may have a direct or indirect relevance to achieving the goal, at certain times or after/at/before certain events.

According to some embodiments, processing circuitry 106 may be in communication with a remote server 107 for tapping into the computing performance thereof, and/or data of previous/other users. According to some embodiments, remote server 107 may be a cloud computer.

According to some embodiments, processing circuitry is designed for a continuous or semi continuous closed loop data input and output, wherein algorithm output and/or device-generated maneuver or stimulation parameters are adjusted based on the input information and data.

According to some embodiments, the output algorithm may be introduced by a new training or education regimen and/or by a device-generated maneuver. It may be introduced to provide an alert for a preferred exercise regimen based on change in the exercise or procedure-relevant parameters from within the human body, for example as a capsule swallowed by the user, or a wearable or any other device placed at certain positions to affect the desired maneuvers or stimulation.

According to some embodiments, the output device may be introduced to provide stimulation or any other performance-relevant signal from within the human body, for example as a transplantable device to be placed at certain positions to affect the desired stimulation or maneuver or an ingestible object (like a capsule).

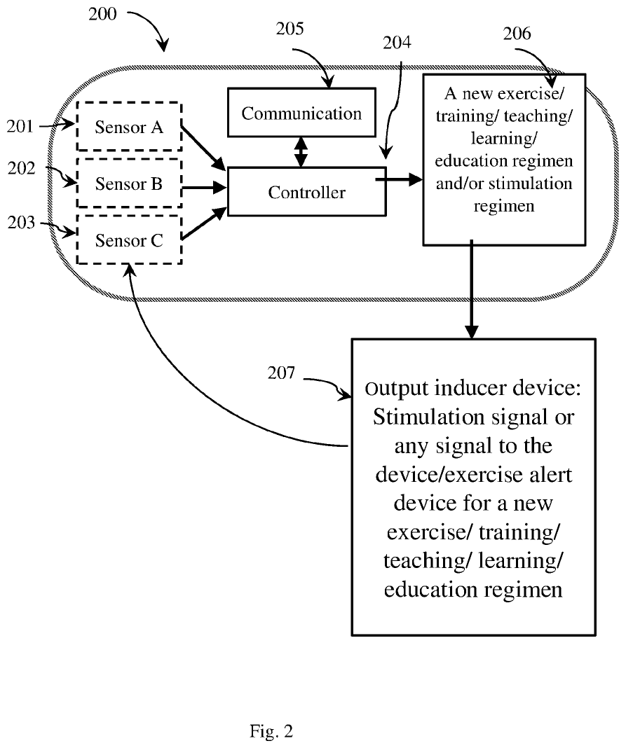

Reference is now made to FIG. 2, which schematically illustrates a functional block diagram of the closed loop-based algorithm for improving an exercise/training/teaching/learning/education regimen and/or stimulation regimen to continuously prevent target organ adaptation and to improve target organ function, and/or loss of response to a regimen, and improving performance, according to some embodiments. According to some embodiments, regimen is in the form of an algorithm that creates alerts for preferred challenged-exercise/training/teaching/learning/education regimens, or combination of regimens, and/or use of device-generated maneuvers of stimulation, or the use of devices or medical devices which generate a procedure that improve organ function, and/or in a form of a pill or any other internal or external device 200, and includes several sensors 201, 202, 203 which collect data. This includes subject-related data and/or organ function related data, and/or exercise/training/teaching/learning/education regimen or disease related data using biomarkers or parameters which are related, or not directly related, to organ function, and to the desired physiological goal, and the pattern of efficacy of a regimen, configured to provide a sum of data to be used for generation of a preferred training regimen and/or a preferred device-generated procedure/stimulation, that continuously prevents adaptation to regular perpetual training methods. The closed loop provides a method for learning and for generating a new exercise/training/teaching/learning/education regimen, and/or maneuver/stimulation or the use of any device which can improve overall target organ or organs function and overall performance, to be delivered to said subject.

The data is being analyzed by the controller via a controller device 204, and a communication device 205. New challenged-exercise/training/teaching/learning/education regimen and/or device-generated maneuver/stimulation regimens are being produced by a device that sums up the data 206.

An output device 207 will continuously generate a new algorithm which is delivered to the subject in the form of a new challenged-exercise/training/teaching/learning/education regimen and/or device-generated maneuver/stimulation regimen for altering the mode of training regimen, and/or device function for target organs. The data of the effect of the output is being re-collected by the sensors 201, 202, 203 and closing the learning loop.

According to some embodiments, device 200 may optionally further include sensors, configured to control the operation of first challenged-exercise/training/teaching/learning/education regimen parameter or device maneuver parameter inducer, as well as several additional such output devices to achieve a physiological change towards a physiological goal, according to exercise/training/teaching/learning/education regimen and/or device-generated maneuver parameters received via the communication unit, which is configured to be in communication with an external or internal update module/unit/circuitry for receiving the parameters, and sending thereto information from the sensors, or other operational information.

According to some embodiments, the output device which continuously generates a new challenged-exercise/training/teaching/learning/education regimen and/or a new device-generated maneuver, may include non-transitory memory for storing exercise/training/teaching/learning/education regimens and device-generated maneuver sessions to be provided to the user. According to some embodiments, the new training regimens and new device-generated maneuvers do not include memory thereon for storing stimulation session, but rather are controlled by the update-unit for continuously changing the regimens' and maneuvers' parameters whenever such a change takes place.

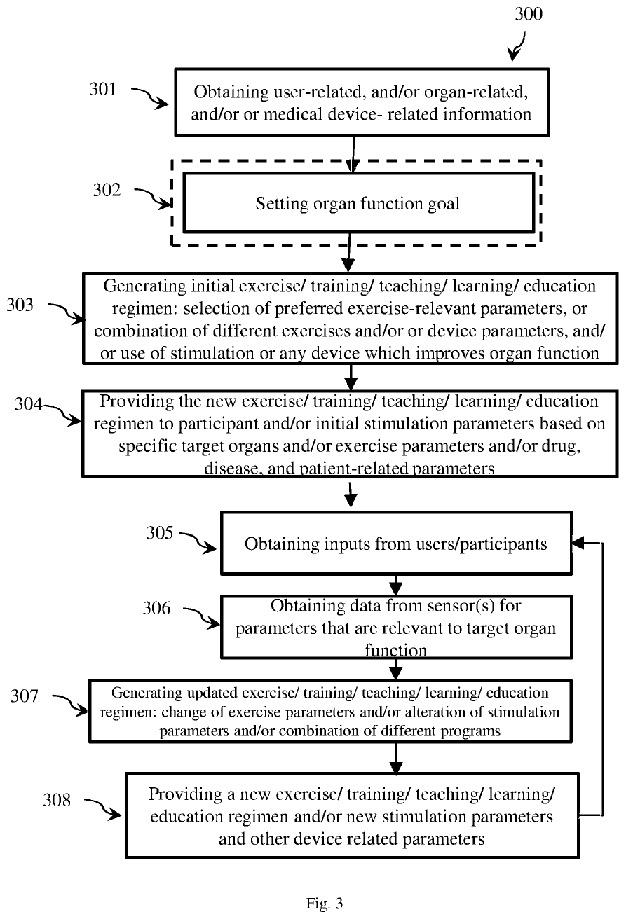

Reference is now made to FIG. 3, which schematically illustrates a flow chart of method 300 for continuously providing updated parameter generation of an alert for a better exercise/training/teaching/learning/education regimen or any type of device-generated maneuver/stimulation signal being generated, according to some embodiments. It schematically illustrates a functional block diagram of the subject-tailored continuously or semi continuously learning-closed loop system. A user related information is obtained (step 301). The user related information may include a sensor measurements, or more general information such as for a non-limiting example subject-related exercise/training/teaching/learning/education regimen-related, organ function-related, disease-related, biomarker-related, and/or any other parameter directly or indirectly of relevance to the effect of the regimen or maneuver on organ performance, such as concomitant medications, scores relevant to organ function, performance of subject, weight, gender, clinical history and the like or data which is specific for organ function or to the exercise. An exercise-specific regimen is determined, an alert is sent to the subjects and a physiological goal is set (step 302). The physiological goal may include a target organ function-related endpoint such as speed of running, heart or lung function, improvement in brain function in Alzheimer's disease, improved learning, amelioration of pain, alleviation of inflammatory disease, malignancy, infection, body weight, glucose levels, blood pressure levels, improvement of function of any organ which is not well functioning, or any organ which is affected by inflammatory, infectious, genetic, or endocrinology, metabolic disease, malignant process, or any other chronic medical condition that requires intervention and/or a positive change of one or more of the above mentioned physiological parameters.

Initial output exercise/training/teaching/learning/education regimen and/or device-generated maneuver/stimulation parameters are determined (step 303). The participant is provided with a new regimen and/or maneuver parameters based on specific target organs and/or regimen parameters and/or drug, disease, exercise-related parameters, and patient-related parameters (step 304). Optionally, input is provided to the subject and to the device, which may include updated target organ function measures, to obtain inputs from the organ or the participant (step 305). Optionally, data from sensor or sensors for parameters that are relevant to target organ performance is obtained (step 306). The updated exercise regimens: change of exercise/training/teaching/learning/education-related parameters and/or alteration of maneuver/stimulation parameters and/or combination of different exercises are generated (307). A new exercise regimens and/or new maneuver or stimulation parameters are provided based on the generated exercise regimen (308) and a closed loop is generated based on updated parameters, and then back to step 305 for new closed loop-based regimens and/or organ maneuver and stimulation regimens.

According to some embodiments, the system may continuously receive input from internal and external devices or from tests, scores, organ-relevant performance parameters, blood tests, or from subject history, from multiple subjects, which is being processed according to deep machine learning closed loop algorithms such that relevant data from other users is being applied to the specific subject to optimize the type of exercise/training/teaching/learning/education regimen. In that way a subject-specific algorithm is generated based on input from the subject and relevant data from other users or subjects.

According to some embodiments, the deep machine learning algorithm is designed to have several levels of closed loops built one on top of the other but also function in parallel to enable the generation of an optimized output algorithm and/or output maneuvers/stimuli, continuously enabling reaching the physiological target or improving organ function.

According to some embodiments, the update system (update module) may have a dual local and network architecture, in which for example the local unit/circuitry is in real-time or short-delay loop with the maneuver device, and learns and updates the maneuver or stimulation parameters without involving a higher-level computational circuitry, such as a server or a cloud computer. The update system may include a global/network component, wherein inputs may be received from multiple users, and learning from the data of the multiple users may be applied in the stimulation parameters of individual users.

Advantageously, in such a local-global architecture, the stimuli may be updated in a short/immediate closed-loop using the lower level (local) update module, wherein longer and less immediate closed-loop may update the stimuli using the higher level (global) update module.

The two-stage hierarchical architecture of the update system brought above is exemplary, and other conceptually similar architectures may apply in various embodiments.

As used herein, the term “update system” or “update module” refers to a component configured to be in wired or wireless communication with the stimulation device for setting and amending algorithm-based regimens and/or maneuver parameters.

According to some embodiments, each data parameter is received and analyzed with correlation to the algorithm-based regimen, and/or maneuver stimuli generated, and thus the algorithm may determine the type of data, or features, which is most relevant for a specific user/subject that correlates with the physiological target or desired physiological change. This input parameter may not be identical to all users/subjects and may not be identical for the same user/subject regarding different physiological targets, objectives, improvements, or desired performances.

According to some embodiments, the algorithm based-challenged-exercise/training/teaching/learning/education/nutritional regimen and/or the device-generated maneuver/stimulation characteristics may change over time even for the same user with the same desired physiological change, and even if, and if not, there is a positive physiological change in organ performance. Such changes in regimens and in maneuvers characteristics may be done for avoiding habituation of the user to the exercise/training/teaching/learning/education/nutritional regimens and device-generated maneuvers, and maintaining a positive physiological change for continuous improved performance, reduced complications, and improved patient adherence.

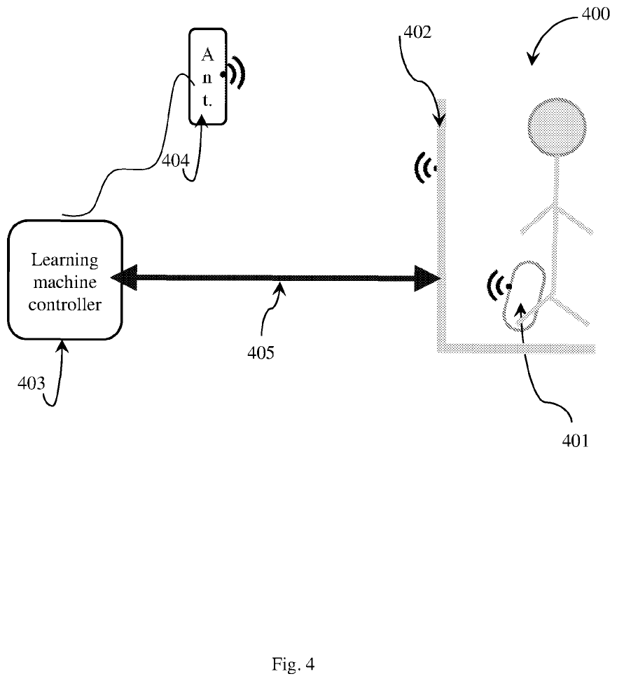

Reference is now made to FIG. 4, which schematically illustrates a person on a treadmill along with a muscle stimulator system 400, according to some embodiments. According to some embodiments, system 400 includes a stimulation device 401, configured to be inserted/introduced to a target area of a subject's legs, to induce stimulation thereto. The treadmill 402 is connected via wireless communication link to the control system 403. According to some embodiments, both the treadmill and the stimulation devices are in communication with an update module, such as a continuous or semi continuous learning machine 403 via wireless communication link, such as through antenna 404, for sending sensor information from treadmill and stimulation devices 401 and 402 to learning machine 403, and receiving updated algorithm-based new exercise regimen and/or stimulation parameters therefrom, 405, to adjust the exercise regimen and/or stimulations for achieving desired results towards reaching an improved target goal of a physiological feature and an ongoing improvement in the running capabilities, reducing complications from the use of a treadmill, and improving subject adherence to the training program.

According to some embodiments, device-generated maneuvers/stimulation techniques include mechanical, magnetic, electric, electromagnetic, ultrasound, thermal or the like which can improve organ function. According to some embodiments, changes in the challenged-exercise/training/teaching/learning/education/nutritional regimens include a change in any parameter of the regimens including for example length of exercise session, degree of difficulty, change in order of independent exercises, and similar parameters, which are of relevance to organ performance. The device-generated maneuvers/stimulation characteristics includes variations or changes in training regimens and in maneuver/stimulation patterns (repetitions), frequency, intensity, and duration or any other parameter that is controlled for these. According to some embodiments, the training and nutritional regimens and device-generated maneuvers may be provided continuously or intermittently with On/Off time periods, and the duration of the time periods and/or the ratio between them may be changed in either a structured manner, randomly or semi-randomly.

According to some embodiments the device is configured to be placed at a desired position on the body of the participant to induce maneuver/stimulation thereto, for example by being fastened using a strap/belt/patch or via any type of a device.

According to some embodiments, challenged-exercise/training/teaching/learning/education/nutritional regimens and maneuver/stimulation devices are in communication with an update module, such as learning machine, for continuously updating regimens and/or maneuvers parameters/characteristics. According to some embodiments, the communication may be wireless.

According to some embodiments, both external and internal devices may be used for data collection and input of data from various organs and/or for the continuous generation of the new challenged-exercise/training/teaching/learning/education/nutritional regimen and the new device-generated maneuver/stimuli required for achieving a target physiological goal and for continuously improving the overall performance of the organ. The closed loop system is continuously or semi continuously receiving data from internal and external measured parameters from one or many users, and is continuously being processed by the controller for generating a new training regimen or a new maneuver/stimuli to be administered to the user via an internal or external device. Optional sensors convey data to the processor that both conveys and is fed data by a cell phone, a cloud and possibly a computer and/or a stimulator device. According to some embodiments, the update-unit/learning-machine is updated upon changes in the measured information, or for example if the change is greater than a certain percentage of the previous value, or if the values reach a predetermined threshold, or any combination of the above.

Disclosed herein is an example of the use of a closed loop continuously learning algorithm for prevention of adaptation for physical exercise.

The target treatment is improved running capability: running more km within a pre-defined period of time and/or reaching a lower pulse.

The physiological target: reaching a lower pulse at the end of the running session.

The exercise regimen algorithm and/or stimulation device (internal or external device) receives data from the sensors (internal and or external), indicative of overall performance of the subject, pulse, breathing, oxygen saturation, blood pressure, skin conductivity along with additional tests and parameters which are relevant or irrelevant to physical activity and to organ or organs' function.

The input data is processed in correlation with the physiological target of the organ function, to assess whether an improvement was achieved, and to what extent, following each exercise period. If no improvement towards the target was achieved a new exercise regimen and/or device-generated maneuver/stimuli is being generated. If a positive step towards the target was achieved the controller will divide each type of exercise regimen (including the degree of difficulty, speed, intervals and additional parameters that are controlled by the algorithm) and/or the selected maneuver/stimuli (electrical, mechanical, magnetic, ultrasound) into 100 percentiles and will determine the percentile for each of the components of the exercise regimen (such as time and degree of difficulty of running within a predetermined range) and/or maneuver/stimuli (such as rate of stimuli, rhythm, power, frequency, amplitude and temperature or others or any combination thereof) and which order of administration or alternating between them was the most efficient in contributing to achievement of the target physiological change that improved the organ performance. Based on that analysis, a continuous new exercise regimen and/or device-generated maneuver/stimuli is generated. In general, the machine learning computer implemented method may require a plurality of samples for learning the user and providing effective stimulations.

The output regimen and/or device generated maneuvers or stimulation parameters update mechanism/algorithm is configured to continuously narrow the range or change the order by which the exercise/training/teaching/education/nutritional regimen is administered, to be targeted on the most effective regimen and/or maneuver/stimulation characteristics for the specific user. However, while narrowing the range for each of the parameters, it will keep the randomization within a predefined continuously changing range.

The output challenged-exercise/training/teaching/learning/education regimen and/or the output device generated maneuvers or stimulation characteristics/parameters update mechanism/algorithm is configured to learn from indications/measurements (measured parameters) which may or may not be directly related to the physiological targets. These include for example any type of parameters which are relevant or irrelevant to said organ function.

According to some embodiments, the algorithm operated in the update module may take into consideration outliers from the plurality of users, to which the learnings of the general users may not fit, and develop new models of treatment (new decision structures) for such outliers.

The algorithm, per one subject, may be developed based on big data analysis generated from multiple subjects. It is noted that the new training regimen and/or the new maneuver/stimuli regimen generated by the big data may be further analyzed by type of organ function, by subject performance, by associated organ, by background diseases, concomitant medications, and subject related factors such as previous scores, tests relevant to the target organ function, age, gender, body weight, delta of change in the target physiological parameter (e.g. running capability) over time, geographic location, and other target organ and/or subject and/or type of training-parameters, it may not be identical per all subjects, and not identical for the same subject under changing conditions. It is only a contributing level of data to the deep machine learning algorithm which generates a subject-specific algorithm.

The output exercise/training/teaching/learning/education/nutritional regimen may be based on a closed loop system in which initially, a plurality of features is received, on which machine learning algorithms are applied. Output parameters are then determined and added as additional feature plurality or used to update the output parameters which are then added as additional feature plurality.

According to some embodiments, the algorithm may change over time per each subject, such that an improvement in the running capability to a certain degree may not require the same training regimen and/or organ maneuver/stimuli that was needed for achieving the previous level. As the algorithm is continuously or semi continuously learning, it will change itself continuously based on both the data being accumulated by the big data and from each subject and other subjects.

For example, the exercise regimen is being generated and delivered by an alert to said subject, altering their speed and degree of difficulty of running, with an alternating pattern of interval times.

For example, a maneuver or stimuli that are being generated by a belt on a muscle, that generate several types of stimuli (electrical, mechanical, vibration and heat) with three stimulation parameters: frequency, intermittency (intervals between On and Off periods), and power/temperature.

For example if a subject suffers from chronic lung disease, or wishes to improve their running capability, and/or lost the effect of treatment and/or is not improving with a regular-perpetual gradual exercise regimen, they can use one of the following or any combination of the following for improving their organ function, prevent loss of the effect of the regular exercise, or for treatment of loss of the effect of the previous exercise regimens, or for maximizing the ongoing effect of the exercise regimen:
a. Use a subject-specific algorithm that determines the irregularity of all parameters of the challenged-exercise/training/teaching/learning/education regimens which are of relevance to the target organ function, by inducing a deep machine learning closed loop algorithm-based irregularity, which is associated with the regimen.
b. Use a maneuver/stimulation-generating device that can be put on the target organ or on any other organ that delivers any type of mechanical, electrical, ultrasound-based, temperature-based, or any other type of stimuli in addition to the training regimen.
c. Use of an algorithm of any combination of the above.

According to some embodiments, the method/system disclosed herein may be used for improvement of function of any tissue/organ/organs in the body including muscle, heart, lung, brain, nerves, kidney, liver, and for improving their performance under all conditions for a continuous achievement of better tissue/organ/organs function, or for treatment of obesity, skin disorders, hair removal, infectious, metabolic, endocrinology, malignant, immune-mediated, inflammatory condition, inborn error of metabolism, pain, microbiome-related disorders, neurological disease, fibrosis in any organ, any type of disease in which circadian rhythm is relevant for, and when a subject wishes to improve the function of its organ, or any type of chronic problem that requires improving the function of tissue/organ/organs.

According to some embodiments, the closed algorithm which receives input from a subject, or groups of subjects, may be utilized for determining a possible change of challenged-exercise/training/teaching/learning/playing/e-sport and any software-related games/education/nutrition regimens including a change of any parameter which is relevant to the improvement of the target or non-target tissue/organ/organs function by these regimens. Any type of input received from the subject or groups of subjects, and assessed by the algorithm for providing an output that may improve these regimens or any type of device-generating maneuvers/regimen/stimulation-based regimen for a subject. This can be applied for any type of regimens aimed at improving the function of a tissue/organ/organs. The challenged-exercise/training/teaching/learning/education/nutrition regimens-based algorithms continuously or semi continuously change the parameters within a predefined range, to improve responsiveness.

According to some embodiments, the challenged-exercise/training/teaching/learning/playing in software-related games/sport/education/nutrition regimens and/or device-generating maneuvers/stimulation is provided for achieving a desired physiological change, and the learning machine success factor is continuously improving and maintaining of a physiological change and/or keep improving the physiological change over time.

According to some embodiments, the goal is improving organ function by exercise/training/teaching/nutrition or education-regimens preventing, or treating or overcoming adaptation or partial/complete loss of an effect of these regimens, or lack of maximal beneficial response to these regimens, enabling a continuous improvement in the performance of the tissue/organ/organs.

According to some embodiments, the method/system disclosed herein may be used for improvement of function of an organ, whether healthy or not, and for continuous improving and reaching a better physiological target in the function of the tissue/organ/organs, or for treatment of obesity, skin disorders, infectious, metabolic, endocrinology, malignant, immune-mediated, inflammatory condition, inborn error of metabolism, pain, microbiome-related disorders, neurological disease, fibrosis in any organ, any type of disease in which circadian rhythm is relevant for, and/or any type of condition which requires improvement of the function of an organ, or chronic problem that requires therapy.

According to some embodiments, the system disclosed herein may include an output system for improving organ performance that can improve an effect of training-regimens and/or devices or maneuvers, which include for example: treadmills, bicycles, muscle building devices, fitness devices, ellipticals, rowers, roman chair, stair master, trampoline, pole, any type of gym device, gym machine, gym equipment, using simulators for education, pilot and driving simulators, teaching devices such as simulators for learning languages, or mathematics, or any maneuver or device which can be used for training, exercising, or education of the organ(s) aimed at improving its function on a single or continuous basis, playing in all types of software-associated games including e-sport, and computer-based games, as well as any type of cosmetic device for hair removal, treatment of any type of skin problem, acne, and rejuvenation and/or devices that generate any type of signals/maneuvers/stimulation inducers, which are configured to affect a the performance of an organ by providing any type of a signal to a target body part, by mechanical signal, physical movement, by electric signal, laser-based device, heat-based devices, and any type of energy produced by a device and being delivered to the organ, by electromagnetic signal emission, by temperature alteration, by using electrical, mechanical, ultrasound wave, or other types of direct or indirect stimuli/signals, by using various types of rate and rhythms of stimuli with various frequencies, amplitudes, durations, and intervals, in structured or random manner or any change in any parameter relevant to the devices.

According to some embodiments, the sensor may be configured to measure, any physiological or pathological parameters that can be measured whether directly or indirectly associated with the physiological target.

According to some embodiments, an alert may be delivered via a cloud based alert system, for example, in real time, such that the alert is connected to any type of partial/complete loss of an effect, of regimens and/or device treatment or maneuver. Such device treatment, may include a medical treatment performed or facilitated by utilizing a medical device. The device treatment or maneuver may also include the use of bicycles, muscle building devices, fitness devices, ellipticals, rowers, any type of gym device, simulators and software for education or for games, as well as any type of cosmetic device for hair removal, treatment of any type of skin problem, acne, and rejuvenation, pilot and driving simulators, teaching devices such as computers and simulators, methods and simulators for learning languages, or mathematics, or any maneuver/procedure/device which can be used for training/exercising/teaching/education/nutrition of an organ or organs aimed at improving its/their function, and/or devices that generate any type of signals/maneuvers/stimulation inducers, which are configured to affect the performance of an organ.

According to some embodiments, the methods and systems disclosed herein may include using an algorithm that prevents any type of complication such as stress fractures, exercise induced asthma, mental responses to training or education, or any other type of complication; and continuously improving the adherence to training regimens. According to some embodiments, the methods and systems disclosed herein may allow continuously improving the response of the organ to these regimens and/or to maneuver or method or device used for a continuous improvement of organ function.

According to some embodiments, the methods and systems disclosed herein may include using an algorithm for improving cognitive and/or mental abilities of subjects with neurological or mental diseases such as Alzheimer. According to some embodiments, the methods and systems disclosed herein may allow continuously improving adherence to training regimens and optionally improving the response of the subject to these regimens and/or to maneuver or method or device being used for a continuous improvement of mental and or cognitive function.

According to some embodiments, the methods and systems disclosed herein may include using an algorithm to improve the long term continuous adherence of said subject to a training-regimen and/or to a device-based training, and their response to any type of partial/complete loss of an effect to any maneuver or method or device being used for improving organ/organs' function.

According to some embodiments, the methods and systems disclosed herein may include an output system/device for improving organ performance by improving the effect of training-regimens and/or devices or maneuvers, which include for example: breeding plant lines, breeding specific animal lines for food, improving efficiency of any type of production line which involves living organisms.

According to some embodiments, the output system/device may be used for improving organ performance by improving the effect of a regimens and/or devices or maneuvers, for example: a woman undergoing a fertility treatment will receive a box with multiples dosages and types of medication aimed at activating the relevant hormonal pathways. The use of this system prevents end organ adaptation to the therapy at a receptor or post receptor-dependent mechanism. Using the algorithm may shorten the treatment and provide the ability for successful therapy through less treatment cycles.

According to some embodiments, the output system/device may be used for improving organ performance by improving the effect of training-regimens and/or devices or maneuvers, for example: subjects may use sport shoes, or any type of sport device, which does not fit their biomechanics, thus taking them out of their “natural comfort zone”, which is increase adaptation. By replacing the shoes between shoes that do not fit their biomechanics between each of the training sessions, the trainees can achieve a better long term effect from his training, overcoming adaptation, improving the target organ performance, and achieving long term continuous improvement in accomplishments.

According to some embodiments, the output system/device may be used for improving organ performance by improving the effect of training-regimens and/or devices or maneuvers, for example: subjects may receive regular or irregular alteration in number of calories, calorie composition, changes in the relation between proteins-carbohydrates-fats-minerals-vitamins, number of meals per day or week, time of meal, method of food preparation (e.g., steamed, cooked, fried, etc.), method of nutrient delivery (e.g., mashed food, frozen food, blended food, etc.).

### EXAMPLES

### Example 1: Subjects undergoing routine exercising program in any type of sport activity

For example, for a subject who is using a treadmill: the subject determines the following pre-exercise parameters: Time of total exercise: 15 minutes; Speed range from 4 km/h to 8 km/h; Level of difficulty from 3.5 to 7.5; period of time for alterations between programs: 30 seconds to 90 seconds.

The device provides the trainer with an algorithm which randomly changes within the predetermined ranges for all parameters. The algorithm is being altered with every repeated exercise and also within the exercise period itself. Each of the pre-set parameters is randomly altered within the predefined windows based on a closed-loop algorithm which is subject-tailored.

The end result for the closed loop system may be any of the followings or any combination of them: improved maximal running ability within a certain time, improved maximal pulse or oxygen saturation reached within a certain time and under certain conditions, caloric meter measurements, or any type of improved physiological endpoint which is relevant to the cardiorespiratory system and or to the muscle system.

Earlier achievement of the target physiological-related endpoint, or achievement of a better than expected endpoint by using the algorithm shows that the algorithm is effective in overcoming target organ adaptation which prohibits reaching a maximal effect for the subject, or is associated with a much longer time required for achieving the effect.

In this example a runner on a treadmill ran for 15 minutes at a speed of 10 km/hour at a pre-fixed angle. His pulse increased from 82 to 149 at the end of the 15 minutes.

Under identical conditions he runs for 15 minutes with a change in speed every 20 seconds in a random manner between 7.5 and 10 km/hour. His pulse increased from 80 to 165 at the end of the five minutes.

This example shows that random alteration of the exercise regimen leads to a more profound effect on the target organs, cardiorespiratory and muscular systems. The data suggests that using this type of an algorithm improves the ability to continuously enhance organ function, leading to a better maximal effect to be achieved from the exercise, and within a shorter time for achieving the goal. Moreover, as it requires the trainee to be in a state of constant alertness for changes, subject cannot perform the exercise in a monotonic way, and must stay focused throughout the training, thereby improving his overall brain control of organs, contributing to further improvement in target organ function.

### Example 2: A Patient with Chronic Heart Failure, or Chronic Obstructive Lung Disease, or Following an Acute Ischemic Heart or Brain Event, or Chronic Neurological Disease, Who is Undergoing a Rehabilitation Program

For any device/method/procedure being used, and with every type of rehabilitation program, the suggested algorithm generates a new training-regimen and/or a new device-based regimen, which randomly changes within the predetermined ranges set by the subject or a care giver such as his coach or physician.

The end result is an organ-performance status, an organ-related score, pulse, saturation, caloric meter measurements, lung function test, echocardiography assessment of heart function, or any type of physiological endpoints, and/or clinical endpoints which are relevant to the patient's disease.

Earlier achievement of target disease-related and/or physiological-related endpoints by using the algorithm shows that the algorithm is effective in continuously overcoming target organ adaptation which prohibits reaching a maximal possible effect, or is associated with a much longer time required for achievement of the performance target.

### Example 3: A Patient with Alzheimer's Disease

The patient may undergo a test for his mental capabilities before and after maneuvers and exercises aimed at improving his overall brain function. Mental status testing evaluates memory, ability to solve simple problems and other thinking skills. Such tests give an overall sense of whether a person: is aware of symptoms; knows the date, time, and where he or she is; can remember a short list of words, follow instructions and do simple calculations. The mini-mental state exam and the mini-cog test are two commonly used tests.

Mini-mental state exam (MMSE): During the MMSE, a health professional asks a patient a series of questions designed to test a range of everyday mental skills. The maximum MMSE score is 30 points. A score of 20 to 24 suggests mild dementia, 13 to 20 suggest moderate dementia, and less than 12 indicates severe dementia. On average, the MMSE score of a person with Alzheimer's declines about two to four points each year.

Mini-cog: During the mini-cog, a person is asked to complete two tasks: remember and a few minutes later repeat the names of three common objects; draw the face of a clock showing all 12 numbers in the right places and a time specified by the examiner. The results of this test help a physician determine if further evaluation is needed.

Computerized tests cleared by the FDA: The U.S. Food and Drug Administration (FDA) has cleared several computerized cognitive testing devices for marketing. Some of these are the Cantab Mobile, Cognigram, Cognivue, Cognition and Automated Neuropsychological Assessment Metrics (ANAM) devices. Computer-based tests such as these in addition to the MMSE and Mini-Cog are also used. Computerized tests have several advantages, including giving tests exactly the same way each time. Mood assessment: In addition to assessing mental status, the doctor evaluates a person's sense of well-being to detect depression or other mood disorders that cause memory problems, loss of interest in life, and other symptoms that overlap with dementia.

Interventions for improvement of Alzheimer disease: Studies have shown that when people keep their minds active, their thinking skills are less likely to decline. Games, puzzles, and other types of brain trainings help slow memory loss and other mental problems. One study involved more than 2,800 adults 65 and older. They went to up to 10 hour-long brain-training sessions for 5 to 6 weeks. (http://www.webmd.com/alzheimers/guide/preventing-dementia-brain-exercises#1) The sessions focused on tactics for these skills: memory; reasoning; speed of processing information; learning something new such as a second language or a musical instrument; playing board games with your kids or grandkids. Regular physical exercise may be a beneficial strategy to lower the risk of Alzheimer's and vascular dementia.

However, adaptation occurs to all of the above exercises, preventing the ability to reach a maximal beneficial effect.

In the present example, patients with Alzheimer's disease receive present perpetual regimens for mental and physical exercises. They repeat the tests before and after these exercises. A second group of patients with Alzheimer's disease perform an identical combination of exercises controlled by a patient-tailored deep machine learning closed loop algorithm. The use of the algorithm leads to better maximal improvement in brain function based on the above parameters, within a shorter period of time, and with higher adherence to performance on the tests. The use of the algorithm puts a higher burden on the brain, preventing a monotonic exercise, thereby improving the effect achieved by the training.

### Example 4: Prevention of Adaptation and Improving Efficacy of Strenuous Physical Exercise for Professional Sport Athletes

Professional athletes use regular perpetual exercising regimens for improving their results. In most cases they reach a plateau in their maximal achievements with only modest improvement with any further training exercises. In the present example, professional athletes use a training algorithm which is based on predetermined ranges for their different training exercises, their previous performance, and a preset order for the different exercises.

The irregularity in each of the procedures, and the irregularity determined by the algorithm, for the combination of different maneuvers, and within each of the maneuvers, and between repeated exercises, will lead to improved results enabling them to reach a higher level of performance. The algorithm is setup to receive data on their pre-defined endpoints and previous performance, and is continuously learning during the exercise and between exercises, altering the parameters which are relevant to each of the exercises within a preset window.

The algorithm provides an output that alters in a random subject-specific way, the different parameters relevant to each of the exercises, as well as selection of a preferred exercise, or combination of different exercises, or combinations with devices that improve function of the target muscle or organs, which are relevant to the target organ. An independent algorithm provides an output that provides subject-tailored training regimens and/or produces an internal or external maneuver/stimulation to the relevant muscles or to other organs, to prevent adaptation to exercise. The end result is continually better performance in the ultimate goal of the athlete, achieved within a shorter period of time, and with fewer complications. The requirements for increased alertness during the training due to continual changes and full randomness which occur during the session, require persistent brain alertness, and improve brain control of the target organ, thereby improving target organ function, and the overall performance of the athlete.

### Example 5: A “Smart Bicycle”/“Smart Treadmill” Based on a Subject-Tailored Continuously/Semi Continuously Deep Machine Learning Closed Loop Algorithm

The “smart bicycles” are connected via sensors to the subject. The predetermined range for all parameters relevant to the training session are inserted into the algorithm prior to starting to use it. The subject who is using the algorithm pushes activates a cell phone application which is connected to the bicycle, and provides a training algorithm that alters all of the relevant parameters such as speed, degree of strain and others, which are continuously/semi continuously being updated by the algorithm in real time or not, based on the input received from the sensors and based on the performance of the subject and are designed by the closed loop mechanism.

The algorithm is designed to improve the functions of organs which are of relevance to the training such as muscles, heart, lung, and brain. Using the algorithm-based training leads to improved organ function and improved overall performance within a shorter period of time. It improves the brain-target organ association, as it requires full alertness of all brain areas associated with the cycling, and thereby leading to overall improved performance.

### Example 6: A Software and Method for Teaching Mathematics and Languages

A subject which sits in front of computer or a teacher, is learning mathematics or a new language. The software, or the teacher, are not using the repetitive gradually increasing difficulty/stepwise approach. Instead, a new algorithm-learning regimen which is based on the use a subject tailored approach is set by the algorithm to provide randomness within a predetermined range for each learning task, and changes between sessions and within each session, and is based on the scores and performance of the subject on initial tests and within or after each training period itself.

This type of learning leads to a better effect within a shorter period of time. It increases the adherence to learning, and improves associations between different parts of the brain associated with learning. The overall result of using the algorithm is a continuously enhanced learning capability as measured by objective tests and validated scores.

### Example 7: Using the Algorithm to Mix Two or More Types of Training for Improved Training Efficiency

A person using a treadmill is asked during the training period by an algorithm based-mixing of tasks to add the use of weight lifting for a few seconds or minutes every certain period of time in a subject-tailored continuously developing randomization based algorithm for improving organ function.

A different way of improving the training session is by combining one or more tasks, which are unrelated to the aim of the training. Such that the person running on a treadmill is exposed every certain period of time to a question on the screen in front of them which requires them to solve a mathematical problem, or any other type of question.

Combing two or more types of tasks, which are related or not related to the target organ (e.g. leg muscle, and cardio respiratory system) is contributing to further improvement of target organ function and to shortening the time of the exercise, as well as enhancing brain activity and improving alertness

### Example 8: Using a Plant-Tailored Continuously Developing Randomization Based Algorithm for Improving Plant Growth

An automatic plant watering system which is adapted to provide the plants with a constant amount of water, and/or keep a certain temperature, and/or provide the plant with certain amount of nutrients is a known method for improving efficiency of growth.

However, this method is associated with plant adaptation and reaches a plateau.

In the present example, the watering system is controlled by an algorithm which is pre-set to provide a certain amount of water and nutrients per day/week/month. The algorithm will alter the amount provided in a continuous or semi continuous way such that it prevents adaptation and continuously improves the efficiency of the plant growth.

The same method may be used for improving the growth of animals for food, or for breeding.

### Example 9: Improving the Efficiency of Fertility Methods

Using a subject-tailored continuously developing randomization based algorithm for improving the efficiency of fertility treatments. Such that each cycle of therapy is based on an algorithm-controlled randomization of the therapy, which is pre-determined within a pre-set range by the physician.

The woman undergoing a fertility treatment will receive a box with multiples dosages and types of medication aimed at activating the relevant hormonal pathways. The use of this system prevents end organ adaptation to the therapy at a receptor or post receptor-dependent mechanism. Using the algorithm may shorten the treatment and provide the ability for successful therapy through less treatment cycles.

### Example 10: Improving the Efficiency of Cosmetic Devices

Devices being used for hair removal, fat burning, skin rejuvenation, anti-aging, acne, or any type of cosmetics are limited in their efficiency due to adaptation of the target organ to the device whether it is laser-based, light-based, or any type of electric-based stimuli, or any other type of stimuli, irrespective of the mechanism used for hair removal, fat cell burning, skin improvement.

In the present example, the algorithm provides an output that alters in a random subject-specific way, the different parameters relevant to each of the treatments, whether using one or more types of mechanisms applied to the patient skin (light, laser, electrical stimuli), as well as selection of a preferred combination of different type of stimuli, by changing the energy source, and/or the range of the stimuli, and/or the rate or rhythm of administration of the stimuli, for improvement of the end result on the target skin or fat or any other target organ, or tissue, or which are relevant to the target organ. An independent algorithm provides an output that provides subject-tailored treatment regimens and/or produces an internal or external maneuver/stimulation to the relevant tissues, to prevent adaptation and improve the efficiency of treatment. The end result is continually better performance, achieved within a shorter period of time, and with fewer complications.

### Example 11: Computer Gaming and e-Sport Gaming

Any type of games whether computer based, or software based, including e-sport games are limited by their ability to generate a monotonous type of brain activation, which leads to adaptation to the tasks required in the game, and limit the ability for improvement, as well as for the joy from the game, and/or the ability to reach maximal effect.

In the present example, the algorithm provides an output that alters in a random subject-specific way, the different parameters relevant to each of the games, as well as selection of a preferred game-related tasks, or combination of different tasks, or combinations with devices that improve function of the target organs required to be activated or function during the game, such as the brain and specific muscles, which are relevant to the game. An independent algorithm provides an output that provides subject-tailored playing regimens to prevent adaptation to the game. The end result is continually better performance, achieved within a shorter period of time, and with increase joy and benefit from the game. The requirements for increased alertness during the game due to continual changes and full randomness which occur during the playing session, require persistent brain alertness, and improve brain control of the target organs relevant to the game, thereby improving target organ function, and the overall performance of the subject.

### Example 12: Improving Dietary Habits and Chronic Body Disease States

Diets and devices that help subjects lose weight are limited in the scope of sustaining weight loss due to brain adaptation to vagal nerve signaling from the stomach wall.

In the present example, the algorithm provides an output that alters in a random subject-specific way, the different parameters relevant to each of the diets/devices/treatments for overweight or obesity. Subjects may receive regular or irregular alteration in number of calories, calorie composition, changes in the relation between proteins-carbohydrates-fats-minerals-vitamins, number of meals per day or week, time of meal, method of food preparation (e.g., steamed, cooked, fried, etc.), method of nutrient delivery (e.g., mashed food, frozen food, blended food, etc.).

### Example 13: Making Better Devices for Training and Learning

While biomechanics are associated with the development of better training devices such as shoes for professional and nonprofessional athletes, to improve their performances by tailoring the shoe to the biomechanics of their leg, and similarly by tailoring a learning software or any other type of training or learning device or program to the trainee, these keep the trainee in the best “comfort zone” for him or her, further leading to adaptation to the exercise, thus reaching a plateau in the achievements.

In the present example, it is suggested to prepare the trainee a shoe, or any type of device, or learning or training program which are opposing many of the features that fits him, thus taking him out of the comfort zone. Subjects may receive regular or irregular alterations in any type of parameter related to the device such as the shoe they use. The shoe will be altered all the time such as by replacing the training shoe between working sessions, or by altering between learning program, or any type of training regimen. These continuous alterations will lead to prevention of adaptation thus leading to a continuous improvement in the overall performance of the trainee. For any type of training, it will improve the target organ such as heart, lung, muscle, nerve, association with the brain. This type of training will lead to better long term effect within a shorter period of training time.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

1.-36. (canceled)

37. A computer implemented method for determining an optimal subject-specific treatment regimen, the method comprising:
receiving a plurality of physiological and/or pathological parameters related to the subject;
applying a closed loop machine learning algorithm to the plurality of physiological and/or pathological parameters;
determining subject-specific treatment regime based on the output parameters wherein the subject-specific treatment regime is selected from a medical treatment regimen, challenged-exercise regimen, training regimen, learning regimen and nutritional regimen; and
optimizing the subject-specific treatment regime by applying a subject-tailored continuously or semi continuously, at least partially randomization-based algorithm to the subject-specific output parameters, wherein the optimized subject-specific treatment regime prevents or mitigates cell, tissue and/or organ adaptation to a treatment regimen and facilitates continual improvement of cell, tissue and/or organ function and/or performance.

38. The method of claim 37, wherein the subject-tailored continuously or semi continuously algorithm is further configured to use or combine one or more algorithm training tasks related to the target cells, tissue, organ and/or body for improving function and/or performance thereof.

39. The method of claim 37, further comprising, utilizing a stimulation device, providing to the subject stimulation for maximizing the effect of the at least one regimen.

40. The method of claim 37, further comprising updating at least one of the subject-specific output parameters.

41. The method of claim 40, wherein updating comprises updating amplitude, frequency, interval, duration of the at least one of the subject-specific output parameters or any combination thereof.

42. The method of claim 40, wherein updating comprises updating amplitude, frequency, interval, duration of the at least one of the subject-specific output parameters or any combination thereof.

43. The method of claim 37, further comprising determining stimulation parameters.

44. The method of claim 37, wherein the output parameters are updated based on data being continuously or semi continuously collected from the subject.

45. The method of claim 37, wherein the machine learning algorithm further considers personal data selected from a group consisting of: subject performance, cell/tissue/organ function-related scores, parameters relevant to cell/tissue/organ performance, age, weight, waist circumference, target organ, and other organs' function, caloric intake and output, gender, ethnicity, geography, pathological history/state, temperature, metabolic rate, brain function, health status, heart, lung muscle function, blood tests, and any physiological or pathological biomarkers, a subject's health related parameter or any combination thereof.

46. The method of claim 37, wherein at least one of the physiological and/or pathological parameters is obtained from a sensor.

47. The method of claim 37, further comprising notifying the subject, in real time, of recommended regimen related parameters or changes thereof.

48. The method of claim 37, further comprising utilizing an external, wearable, swallowed and/or implanted device for evoking a reaction in the target cells, tissue and/or organ for continually improving function and/or performance thereof.

49. The method of claim 37, further comprising administering challenged-exercise regimen, training regimen, education regimen, nutritional regimen or device-generated maneuvers regimens to the subject.

50. The method of claim 37, further comprising updating the challenged-exercise/training/teaching/learning/playing/education regimens/nutritional regimens, and/or device generated maneuvers or stimulation parameters, wherein updating comprises utilizing machine-learning capabilities.

51. The method of claim 37, wherein the machine learning capabilities include closed-loop deep learning capabilities.

52. The method of claim 37, wherein the machine learning capabilities are configured to be operated on a set of features by receiving values thereof.

53. The method of claim 37, used for improving organ function in healthy subjects who wish to improve muscle, heart, lung, skin, brain on any other tissue/organ/organs performance, and/or for improving training capabilities of any tissue/organ/organs, improving education, or teaching, and/or for treatment of obesity, infectious, metabolic, endocrinology, malignant, immune-mediated, inflammatory condition, inborn error of metabolism, pain, microbiome-related disorders, neurological disease, fibrosis in an organ, desynchronosis or circadian dysrhythmia.

54. The method of claim 37, wherein the treatment comprises a drug treatment, a device treatment or a combination thereof.

55. A system for determining an optimal subject-specific treatment regimen, the system comprising a processor configured to:
receive a plurality of physiological and/or pathological parameters related to the subject;
apply a closed loop machine learning algorithm to the plurality of physiological and/or pathological parameters;
determine subject-specific treatment regime based on the output parameters, wherein the subject-specific treatment regime is selected from a medical treatment regimen, challenged-exercise regimen, training regimen, learning regimen and nutritional regimen; and
optimize the subject-specific treatment regime by applying a subject-tailored continuously or semi continuously, at least partially randomization-based algorithm to the subject-specific output parameters wherein the optimized subject-specific treatment regime prevents or mitigates cell, tissue and/or organ adaptation to a treatment regimen and facilitates continual improvement of cell, tissue and/or organ function and/or performance.

56. The system of claim 55, wherein the subject-tailored continuously or semi continuously algorithm is ongoing developed and is further configured to combine one or more algorithm training tasks related to the target cells, tissue, organ and/or whole body for improving function and/or performance thereof.
